SEQUENCE LISTING (4 sequences)

SEQ ID NO: 1 - protein, 141 aa, Sclerotium rolfsii
TYKITVRVYQTNPNAFFHPVEKTVWKYANGGTWTITDDQHVLTMGGSGTSGTLRFHADNGESFTATFGVHNYKRWCDIVTNLAADETGMVINQQYYSQKNREEARERQLSNYEVKNAKGRNFEIVYTEAEGNDLHANLIIG

SEQ ID NO: 2 - protein, 141 aa, Artificial Sequence
  In vitro synthesis
TYKITVRVYQTNPDAFFHPVEKTVWKYANGGTWTITDDQHVLTMGGSGTSGTLRFHADNGESFTATFGVHNYKRWCDIVTNLAADETGMVINQQYYSQKNREEARERQLSNYQVKNAKGRNFQIVYTEAEGNDLHANLIIG

SEQ ID NO: 3 - protein, 141 aa, Artificial Sequence
  In vitro synthesis
VYKITVRVYQTNPDAFFHPVEKTVWKYANGGTWSITDDQHVLTMGGSGTSGTLRFHADNGESFTATFGVHNYKRWCDIVTNLAADETGMVINQQYYSQKNREEARERQLSNYQVKNAKGRNFQIVYTEAEGNDLHANLIIG

SEQ ID NO: 4 - protein, 141 aa, Artificial Sequence
  In vitro synthesis
VYKITVRVYQTNPDAFFHPVEKTVWKYADGGTWSITDDQHVLTMGGSGTSGTLRFHADNGESFTATFGVHDYKRWCDIVTDLAADETGMVINQEYYSEKDREEARERQNSNYEVKDAKGRNFEIVYTEAEGNDLHADLIIG

(19) United States
(12) Patent Application Publication
(10) Pub. No.: US 2023/0348564 A1
(43) Pub. Date: Nov. 2, 2023

(54) ANTI-CANCER PROTEINS

(71) Applicant: UNICHEM LABORATORIES LTD, Maharashtra (IN)

(72) Inventors: Dhananjay Sathe, Maharashtra (IN); Sarvanakumar Iyappan, Telangana (IN); Dilip Pawar, Maharashtra (IN)

(21) Appl. No.: 17/923,732

(22) PCT Filed: Apr. 28, 2021
(86) PCT No.: PCT/IB2021/053503

§ 371 Date: Nov. 7, 2022

(30) Foreign Application Priority Data
May 7, 2020 (IN) 202021019406

(51) Int. Cl. C07K 14/705; A61P 35/00

(52) U.S. Cl. CPC C07K 14/7056; A61P 35/00; C07K 2317/73; C07K 14/475

(57) ABSTRACT

A recombinant lectin for use in a method of treatment of cancer by inhibiting angiogenesis in a subject. The treatment comprises administration of a therapeutically effective amount of the recombinant lectin.

### FIELD OF INVENTION

The present invention relates to the use of a lectin in the treatment of cancer. In particular, the present invention relates to the use of a lectin protein having an anti-angiogenic and apoptotic effects on cancer cells.

### BACKGROUND OF INVENTION

The active immune system is responsible for the healthy individual; as an immune system defend the several diseases or diseased conditions. It is also believed that the immune system resists even the formation of cancer by destroying cancer cell. When the immune system fails to do so, this can result in the formation of cancer. The word “cancer” describes the number of diseases which are characterized by the unregulated growth and uncontrolled division of abnormal cells. Cancer can arise from virtually to any tissue or organ in the human body. Despite recent developments in medicine and the understanding of the molecular basis of cancer, the exact causes of any given type of cancer are unknown in a particular individual. Given this lack of knowledge, it remains highly difficult to find cancer treatments that would be effective for a particular individual.

Finding effective cancer treatment is also made challenging because cancer often develops resistance to various therapeutic strategies in an individual. Moreover, an effective means for treating cancer may become less effective as certain types of cancers can spread from their primary source or origin. This process, called metastasis, enables cancer cells to spread to other vital parts of the body through the blood and lymph systems.

Moreover, other challenges faced in the treatment of these metastasized cells in the destination tissue, include metastasized cells surviving in the destination tissue by overcoming local immune defenses, and acquiring their own blood supply and nutrients through the process of angiogenesis. Nevertheless, metastasis remain a key reason why effective cancer treatments are difficult to develop.

Existing cancer therapies today include various ablation techniques such as surgical procedures; cryogenic or heat methods on the tissue, ultrasound, radiofrequency, and radiation; chemical methods such as pharmaceuticals, cytotoxic agents, monoclonal antibodies; or trans-arterial chemo immobilization (TACE), and combinations thereof according to specific regimens based on the specific type and stage of cancer under treatment. However, these therapies are associated with substantially high costs. In addition, current treatment options are highly invasive with significant toxicity and side effects, and result in an overall poor quality of life for patients.

Specificity towards the malignant cells helps to avoid damage to healthy cells and lessen the toxicity associated with the therapy. During malignant transformation and metastasis, glycans are modified due to altered pathophysiological condition and altered glycosylation by cancer cells. These modified glycans can be easily detected by the Glycan specific binding protein or tumor specific lectin protein (hereinafter, lectin). Moreover, these proteins also play a significant role in decoding the information related to glycans. Lectins are naturally occurring carbohydrate binding proteins; which can specifically detect the cancer associated antigen due to altered glycosylation. Because of their unique ability and specificity, lectins are useful in diagnostic and therapeutic purposes.

Oncofetal Thomsen-Friedenreich antigen (Galβ1-3GalNAc-α-O-Ser/Thr, T or TF), which is expressed in more than 90% of human cancers and is correlated with tumor progression and metastasis. The Applicant's previous patent application WO2010/095143 discloses a lectin isolated from fungus Sclerotium rolfsii having high binding specificity towards TF.

Patent Number CN106397554 describes a preparation and application method to provide Cordyceps millitaris (CCM) lectin protein. Lectin-CCM and its anti-proliferation activities tested in vitro on human cervical HeLA cancer cell line.

Patent Number KR1020030091386 describes a process of preparation of an extract of Korean mistletoe (Viscum album coloratura) with a lectin ingredient in an extract. When administered to a mouse experimental model, the lectin showed enhanced antitumor and anti-metastatic activity.

Recombinant mistletoe lectins have been used for the treatment of skin cancer, in particular malignant melanoma in the form of metastatic tumor (Stage III and Stage IV). The Patent Number RU0002639445 mentions a pharmaceutical composition, containing recombinant mistletoe lectin, for treatment of melanotic cancer. Treatment with recombinant mistletoe lectins is known to extend significantly the survival rate of cancer patients.

U.S. Pat. No. 7,045,300 describes a lectin protein, MFA (Maackia fauriei agglutinin) extracted from the Korean legume Maackia fauriei, its used as a diagnostic agent for cancers and therapeutically used as an anti-proliferation agent (or an anti-cancer agent) in diseases in which N-acetylneuraminic acid exists, in particular breast cancer, melanoma or hepatoma.

U.S. patent Ser. No. 10/294,295 describes a method of treatment of cancer by modulating angiogenesis with VEGF antagonist, in particular, galectin-1 sequences have been used as VEGF antagonist to inhibit the angiogenesis for the treatment of cancer.

Patent Number KR1020030028855 describes an anticancer composition containing an extract of Korean mistletoe (Viscum album coloration) with a lectin as an active ingredient for inhibition of metastases via inhibiting angiogenesis and inhibition of telomerase activity.

Even though there are few reports of lectin showing efficacy as anti-tumor agents, they are not well explored. Lectins show high specificity, less cytotoxicity and are easy to synthesize on a larger scale. It is the need of the hour to develop lectins as new, cheap and better method of treatment of tumors. It is essential to know the molecular mechanism of action of the lectins or combination of lectin with any anti-cancer drugs to enhance their therapeutic potential.

The object of the present invention is to study and develop lectins so as to make them available as anti-tumor agents.

### SUMMARY OF INVENTION

According to an aspect of the invention there is provided a recombinant lectin for use in a method of treatment of cancer.

According to another aspect of the invention there is provided a recombinant lectin for use in a method of treatment of cancer by inhibiting angiogenesis in the cancer cell comprising administration of therapeutically effective amount of a recombinant lectin protein.

According to yet another aspect of the invention, there is provided a recombinant lectin for use in a method of treatment of cancer by inducing apoptosis in the cancer cell comprising administration of therapeutically effective amount of a recombinant lectin protein. According to this aspect the lectin induces early and late stage of apoptosis in the cancer cells.

According to yet another aspect of the invention, there is provided a recombinant lectin protein as an angiogenesis inhibitor and/or an apoptosis inducer, thereby prohibiting cancer cell metastasis.

Apotosis is the process for the programmed cell death by the signalling pathways. The term “inducing apoptosis” means herein activating signalling pathways responsible for the programmed cell death of the tumor cells.

Metastases is the spread of the cancer cells from their primary source to other vital parts of the body through the blood and lymph systems. The term “prohibiting metastases” means herein decreasing the metastases from primary source or origin of cancer by reducing the spread to the vital organs and body parts.

In another aspect of the invention, there is provided a method of treatment of cancer by inhibiting angiogenesis in the cancer cells wherein the method comprises administration of therapeutically effective amount of a recombinant lectin protein to a subject.

In yet another aspect of the invention, there is provided a method of treatment of cancer by inducing apoptosis in the cancer cells, wherein the method comprises administration of therapeutically effective amount of a recombinant lectin protein to a subject.

According to an aspect of the invention, there is provided a pharmaceutical composition for use in the method of treatment of cancer comprising therapeutically effective amount of a recombinant lectin protein and a pharmaceutically acceptable excipient, wherein the composition inhibits angiogenesis in the cancer cells.

According to yet another aspect of the invention, there is provided a pharmaceutical composition for use in the method of treatment of cancer comprising therapeutically effective amount of a recombinant lectin protein and a pharmaceutically acceptable excipient, wherein the composition induces apoptosis in the cancer cells.

According to yet another aspect of the invention, there is provided a method of preventing angiogenesis in tumor cells using therapeutically effective amount of a recombinant lectin protein.

According to yet another aspect of the invention, there is provided a method of inducing apoptosis of tumor cells using therapeutically effective amount of a recombinant lectin protein.

According to the preceding aspects of the invention the cancer is a carcinoma such as adenocarcinoma or squamous cell carcinoma.

According to the specific aspect of the present invention the adenocarcinoma is a carcinoma of oesophageal, pancreatic, prostate, cervical, breast, colon or colorectal, lung, bile duct, vaginal, urachus or stomach adenocarcinoma.

According to the specific aspect of the present invention, the squamous cell carcinoma is the cancer of squamous cells of skin, lung, oral, thyroid, oesophagus, vaginal, cervical, ovarian, head and/or neck, prostate or bladder.

According to yet another specific aspect of the present invention, the cancer is brain cancer.

According to any one of the preceding aspect of the invention, the effective concentration of recombinant lectin protein is from about 0.1 μg/mL to about 200 μg/mL.

According to any one of the preceding aspect of the invention, the therapeutically effective dose of recombinant lectin protein is from about 0.1 mg/Kg to about 100 mg/Kg body weight of a subject.

According to further aspect of the present invention, the recombinant lectin inhibits migration and/or proliferation of endothelial cells, modulates VEGF secretion and reduces hemoglobin content and neovasculization in the cancer cells.

According to any one of the preceding aspect of the invention, the recombinant lectin modulates one or more markers or signaling pathways selected from: ATF-2, ERK1/2; JNK; MEK-1; P90RSK; STAT-3; p53; MMPs; HGF; C-kit; Her-2; GMSCF; IL-6; IL-8; p38/MAPK; PDGF; TNFR; MPO; Galectin-3; Fol-1; CD40L; Angiopoietin-2; Kalikrein-5; Osteopontin; TNF-α; Endoglin; MAPK/EGFR/Ras/Raf; ADBR1; CCR5; IL-4/STAT6; NF-KB; PI3K/AKT/FOXO3; PKC/CA2+; and TNF-α/JNK, TRAIL via FADD caspase-3, Leptin, Contactin-1, Notch-1 and HGFR/c-MET.

According to any one of the preceding aspects, the recombinant lectin is represented by an amino acid sequence having at least 60% identity to SEQ ID NO. 1 or the amino acid sequence having at least 70%, 80%, 90%, 95%, 96%, 97%, 98% or 99% homology to SEQ ID NO. 1. According to the specific aspect the recombinant lectin is selected from the amino acid sequence having SEQ ID NO. 2, SEQ ID NO. 3 or SEQ ID NO. 4.

According to any one of the preceding aspects, the recombinant lectin is a modified lectin protein (i.e. a recombinant lectin protein having at least one amino acid modification in a carbohydrate binding site) as defined in WO2020/044296 which is incorporated herein by reference, in particular with regard to the definition of the lectin. In a specific aspect, the recombinant lectin comprises at least one amino acid modification in a carbohydrate binding site of SEQ ID NO. 1 or an amino acid sequence having at least 60% homology to SEQ ID NO. 1.

In another specific aspect, the carbohydrate binding site is a primary and/or secondary carbohydrate binding site.

In another specific aspect, the primary carbohydrate binding site comprises a position selected from 1 or more of 27, 28, 47, 48, 70, 71, 72 & 105 in SEQ ID NO. 1 or in an amino acid sequence having at least 60% homology to SEQ ID NO. 1.

In another specific aspect, the position of the amino acid modification is selected from one or more of:
i) 27 and/or 28;
ii) 47 and/or 48;
iii) 70, 71, and/or 72; and/or
iv) 105.

In another specific aspect, the secondary carbohydrate binding site comprises a position selected from one or more of 77, 78, 80, 101, 112, and 114 in SEQ ID NO. 1 or in an amino acid sequence having at least 60% homology to SEQ ID NO. 1.

In another specific aspect, the position of the amino acid modification is selected from one or more of:
i) 77, 78, and/or 80;
ii) 101; and/or
iii) 112, and/or 114.

In another specific aspect, the amino acid modification is an amino acid substitution such that a substituting amino acid replaces an original amino acid.

In another specific aspect, the amino acid substitution in the primary carbohydrate binding site is selected from one or more of:
i) at position 27: a conservative, favourable or unfavourable amino acid, wherein the conservative amino acid is non-polar or acidic; favourable is polar or basic and unfavourable amino acid is non-polar;
ii) at position 28: a conservative, favourable, neutral or unfavourable amino acid, wherein the conservative amino acid is non-polar; favourable is polar, neutral is acidic or basic and unfavourable amino acid is polar;
iii) at position 47: an unfavourable amino acid, which is basic or non-polar;
iv) at position 48: an unfavourable amino acid, which is non-polar;
v) at position 70: an unfavourable amino acid, which is non-polar;
vi) at position 71: an unfavourable amino acid, which is non-polar;
vii) at position 72: an unfavourable amino acid, which is non-polar; and/or
viii) at position 105: a conservative, favourable, neutral or unfavourable amino acid, wherein the conservative amino acid is basic or non-polar; favourable is polar, neutral is acidic, basic or polar and/or unfavourable amino acid is polar, non-polar or acidic.

In another specific aspect, the amino acid substitution in the secondary carbohydrate binding site is selected from one or more of:
i) at position 77: an unfavourable amino acid which is non-polar;
ii) at position 78: an unfavourable amino acid which is non-polar;
iii) at position 80: an unfavourable amino acid which is non-polar;
iv) at position 101: a favourable, an unfavourable or a neutral amino acid, wherein the favourable amino acid is polar or basic, the unfavourable amino acid is non-polar and the neutral amino acid is non-polar or acidic;
v) at position 112: an unfavourable amino acid which is non-polar;
vi) at position 114: an unfavourable amino acid which is polar.

In another specific aspect, the lectin protein comprises at least one amino acid modification in the N-terminus of SEQ ID NO.1 or in an amino acid sequence having at least 60% homology to SEQ ID NO. 1, wherein the N-terminus comprises a position selected from: 1 and/or 2 in SEQ ID NO. 1 or a corresponding position in the sequence having at least 60%, 70%, 80%, 90%, 95%, 97% or 99% homology thereto.

In another specific aspect, the amino acid modification is an amino acid substitution at position 1 and wherein a substituting amino acid is not threonine or valine.

In another specific aspect, the substituting amino acid is selected from: alanine, glycine, proline or serine.

In another specific aspect, the amino acid modification is an amino acid substitution at position 2 and wherein a substituting amino acid is tryptophan.

In another specific aspect, cleavage of an initiator methionine is increased or decreased as compared with a control.

In another specific aspect, the amino acid modification at position 76 is an amino acid substitution with a non-polar amino acid.

In another specific aspect, the non-polar amino acid is selected from: glycine, valine or leucine.

In another specific aspect, the amino acid modification at position 44 or 89 is an amino acid substitution with a non-polar amino acid.

In another specific aspect, the non-polar amino acid is selected from: leucine, isoleucine or valine.

In another specific aspect, the modified lectin protein is soluble, partially soluble or insoluble and/or has cytotoxicity.

In another specific aspect, the modified lectin protein has a cytotoxicity that is at least 10%, 20%, 30%, 40%, 50%, 60%, 70%, 80%, or 90% of a control.

In another specific aspect, the modified lectin protein has a percentage cytotoxicity that is less than 10% of a control, or is absent of cytotoxicity.

In another specific aspect, the modified lectin protein has a percentage cytotoxicity that is at least a 10%, 20%, 30%, 40%, 50%, 60%, 70%, 80%, 90%, or 100% increase compared with that of a control.

In another specific aspect, the modified lectin protein is equal to or less than 500, 400, 300, 250, 200, or 150 amino acids in length.

In a specific aspect, the present invention provides a method of preventing angiogenesis in tumor cells using therapeutically effective amount of a recombinant lectin having the amino acid sequence of SEQ ID NO. 2.

In another specific aspect, the present invention further provides a method of inducing apoptosis of tumor cells using therapeutically effective amount of a recombinant lectin having the amino acid sequence of SEQ ID NO. 2.

In yet another specific aspect, the present invention provides an effective anti-angiogenesis using from about 0.1 mg/Kg to 100 mg/Kg body weight of a recombinant lectin having the amino acid sequence of SEQ ID NO.2.

The present invention further relates to effective anti-angiogenesis in tumor cells using from about 0.1 μg/mL to 200 μg/mL concentration of a recombinant lectin having the amino acid sequence of SEQ ID NO. 2.

The present invention further relates to effective apoptosis using from about 0.1 mg/Kg to 100 mg/Kg body weight of a recombinant lectin having the amino acid sequence of SEQ ID NO. 2.

The present invention further relates to effective apoptosis of tumor cells using from about 0.1 μg/mL to about 200 μg/mL concentration of a recombinant lectin having the amino acid sequence of SEQ ID NO. 2.

In yet another specific aspect, the present invention provides a method of treatment of adenocarcinoma, squamous cell carcinoma and/or brain cancer by preventing angiogenesis and/or by inducing apoptosis in tumor cells using a recombinant lectin having the amino acid sequence of SEQ ID NO.2.

In yet another specific aspect, the present invention provides a method of treatment of adenocarcinoma, squamous cell carcinoma and/or brain cancer by preventing angiogenesis and/or by inducing apoptosis using from about 0.1 mg/Kg to 100 mg/Kg body weight of a recombinant lectin having the amino acid sequence of SEQ ID NO. 2.

In yet another aspect, the present invention provides a method of treatment of adenocarcinoma, squamous cell carcinoma and/or brain cancer by preventing angiogenesis and/or by inducing apoptosis in the tumor cells using from about 0.1 μg/mL to about 200 μg/mL concentration of a recombinant lectin having the amino acid sequence of SEQ ID NO.2.

The present invention also relates to the evaluation of in vitro apoptotic effect of a recombinant lectin having the amino acid sequence of SEQ ID NO.2 in a breast cancer cell line, a colon cancer cell line, a pancreatic cancer cell line and brain cancer cell lines.

The present invention further relates to the evaluation of the modulatory effect of a recombinant lectin having the amino acid sequence of SEQ ID NO.2 on key signaling pathways involved in the pathogenesis of cancer.

The present invention further relates to the evaluation of the anti-tumor potential of a recombinant lectin having the amino acid sequence of SEQ ID NO. 2.

In a further aspect, the present invention relates to a recombinant lectin having the amino acid sequence of SEQ ID NO. 2 as an angiogenesis inhibitor and/or an apoptosis inducer, thereby prohibiting cancer cell metastasis.

### BRIEF DESCRIPTION OF THE ACCOMPANYING SEQUENCES

SEQ ID NO: 1: represents the native S. rolfsii lectin amino acid sequence.

SEQ ID NO: 2: represents a variant of the S. rolfsii lectin amino acid sequence (reported as Rec-2 in WO 2010/095143).

SEQ ID NO: 3: represents a variant of the S. rolfsii lectin amino acid sequence (reported as Rec-3 in WO 2010/095143).

SEQ ID NO: 4: represents a variant of the S. rolfsii lectin amino acid sequence (reported in WO 2014/203261).

### DETAIL DESCRIPTION OF INVENTION

The term “lectin” as used herein refers to a carbohydrate-binding protein.

The term “protein” as used herein refers to a polymer of amino acid residues.

The term “amino acid” as used herein refers to naturally occurring and synthetic amino acids, as well as amino acid analogues and amino acid mimetics that have a function that is similar to the naturally occurring amino acids. Naturally occurring amino acids are those encoded by the genetic code and include the proteinogenic amino acids. Naturally occurring amino acids also include those modified after translation in cells. Synthetic amino acids include non-canonical amino acids such as selenocysteine and pyrrolysine. Typically synthetic amino acids are not proteinogenic amino acids.

It is understood that amino acids can be grouped according to different biochemical properties. Examples include: the polar amino acids, the non-polar amino acids, the acidic amino acids and the basic amino acids. In one embodiment, the amino acid used for the amino acid modification is at least one selected from the group consisting of, but not limited to: polar, non-polar, acidic, basic, selenocysteine, pyrrolysine and non-canonical.

The terms “homology” or “homologous” as used herein refer to two or more referenced entities that share at least partial identity over a given region or portion. Areas, regions or domains of homology or identity refer to a portion of two or more referenced entities that share homology or are the same. Thus, where two sequences are identical over one or more sequence regions they share identity in these regions. Substantial homology refers to a molecule that is structurally or functionally conserved such that it has or is predicted to have at least partial structure or function of one or more of the structures or functions (e.g., a biological function or activity) of the reference molecule, or a relevant/corresponding region or portion of the reference molecule to which it shares homology.

In one embodiment, the percentage “homology” between two sequences is determined using the BLASTP algorithm with default parameters (Altschul et al. Nucleic Acids Res. 1997 Sep. 1; 25(17):3389-402). In particular, the BLAST algorithm can be accessed on the internet using the URL: https://blast.ncbi.nlm.nih.gov/Blast.cgi. In an alternative embodiment, for global sequence alignments, percentage homology between two sequences is determined using the EMBOSS Needle algorithm using default parameters. In particular, the EMBOSS Needle algorithm can be accessed on the internet using the URL: https://www.ebi.ac.uk/Tools/psa/emboss_needle/.

Unless otherwise indicated, the term “homology” is used interchangeably with the term “sequence identity” in the present specification.

The term “recombinant” means a nucleic acid or a polypeptide has been artificially or synthetically (i.e., non-naturally) altered by human intervention. The alteration can be performed on the material within, or removed from, its natural environment or state. For example, a “recombinant nucleic acid” is one that is made by recombining nucleic acids, e.g., during cloning, DNA shuffling or other well-known molecular biological procedures. A “recombinant DNA molecule” is comprised of segments of DNA joined together by means of such molecular biological techniques. The term “recombinant protein” or “recombinant polypeptide” as used herein refers to a protein molecule which is expressed using a recombinant DNA molecule. The recombinant protein according to present invention is the protein having amino acid sequence of SEQ ID 1 which is also referred as SEQ ID 1.

The term “recombinant protein” is intended here to cover any pharmaceutically acceptable salt, solvate, hydrate, prodrug, or any other compound which, upon administration to the patient is capable of providing (directly or indirectly) the compound as described herein. The preparation of salts, solvates, hydrates, and prodrugs can be carried out by methods known in the art.

The terms “effective” or “therapeutically effective” refer to an effect sufficient to elicit the desired biological response. As will be appreciated by those of ordinary skill in this art, the effect of an inventive combination may vary depending on such factors as the desired biological endpoint, the pharmacokinetics of the agents being delivered, the disease being treated, the mode of administration, and the patient. Treatment is generally “effective” if one or more symptoms or clinical markers are reduced. Alternatively, treatment is “effective” if the progression of a disease, disorder or medical condition is reduced or halted.

The term “therapeutically effective amount” as used herein is an amount sufficient to effect desired clinical results (i.e., achieve therapeutic efficacy). A therapeutically effective amount can be administered in one or more administrations. For purposes of this invention, a therapeutically effective amount of a recombinant protein is an amount that is sufficient to palliate, ameliorate, stabilize, reverse, prevent, slow or delay the progression of the disease state.

The term “pharmaceutical composition” or “pharmaceutically acceptable composition” or “pharmaceutically acceptable formulation” refers to a mixture of a compound disclosed herein with pharmaceutical excipients, such as diluents or carriers (see, for example, Remington: The Science and Practice of Pharmacy 22nd ed., Pharmaceutical Press (Sep. 15, 2012) and handbook of Pharmaceutical Excipients, 6th edition, Raymond Rowe, Pharmaceutical Press (2009)). The pharmaceutical composition facilitates administration of the compound to an organism. Pharmaceutical compositions will generally be tailored to the specific intended route of administration.

The term “Signalling pathway” refers to a cascade of chemical reaction in which a group of molecule in a cell work together to maintain processes such as cellular functions, cell differentiation, cell proliferation and cell death. In signal pathway activation/inhibition signal from a biologically active molecule bind to the specific protein receptor on or in the cell and activates the signal. Activation of the first molecule transduces the activation signal to the other molecule and the process is repeated till the cell function is achieved. Abnormal activation of the signalling pathway may lead to diseases such as cancer. By targeting the specific molecule responsible for abnormal signalling pathway, may result in treatment of cancer.

According to a first aspect of the invention there is provided a lectin for use in a method of treatment of cancer.

According to another aspect of the invention there is provided a recombinant lectin for use in a method of treatment of cancer by inhibiting angiogenesis in the cancer cell, wherein the method comprises administration of therapeutically effective amount of a recombinant lectin protein.

As is known in the art, “angiogenesis” is the growth of new blood vessels. Anti-angiogenic agents are known anti-cancer drugs which work by preventing tumours from growing blood vessels. Thus, as used herein, “inhibiting angiogenesis” will be understood as preventing, delaying or reducing the formation of blood vessels. Surprisingly, the present inventors have found that lectins are able to exert an anti-cancer effect by inhibiting angiogenesis.

It will be appreciated that the angiogenesis is prevented/inhibited in a tumour, such as a tumour in a mammalian body, e.g. a human body.

The lectin may be naturally occurring. In an embodiment, the lectin is derived from the group consisting of, but not limited to, fungus and plants.

In some embodiments, the lectin is a fungal lectin. Suitable fungal lectins may be derived from Agaricus bisporus (e.g. ABL), Sclerotium rolfsii (e.g. SRL) and Xerocomus chrysenteron (e.g. XCL).

In some embodiments the lectin is derived from a soil borne phytopathogenic fungus, such as S. rolfsii. By “derived from” it will be understood that the lectin comprises an amino acid sequence which is identical or similar to a native sequence and is synthesized in the laboratory using recombinant DNA technology. The lectin may comprise an amino acid sequence having at least 60% 70%, 80%, 90%, 95%, 96%, 97%, 98% or 99% homology to a native sequence.

The lectin may comprise an amino acid sequence having at least 60% homology to SEQ ID NO: 1. In some embodiments, the amino acid sequence has at least 70%, 80%, 90%, 95%, 96%, 97%, 98% or 99% homology to SEQ ID NO: 1.

In some embodiments, the lectin comprises an amino acid sequence selected from the group consisting of SEQ ID NO: 2, SEQ ID NO: 3 or SEQ ID NO: 4.

The lectin may be recombinant, or it may be synthesized de novo. Methods for preparing recombinant proteins will be well-known to those skilled in the art. For example, a recombinant DNA molecule, such as a plasmid or viral vector, comprising a nucleic acid sequence encoding the lectin may be provided. The nucleic acid sequence may be operatively linked to a promoter which is capable of controlling expression of the lectin in a suitable host cell. The recombinant DNA molecule may be inserted into a suitable host cell using methods known in the art, for example by transformation. Suitable host cells include prokaryotic cells (e.g. E. coli) and both lower eukaryotic cells (e.g. yeast cells) as well as higher eukaryotic cells. The host cell can then be cultured under appropriate conditions, whereby the recombinant lectin is expressed. The recombinant lectin can thus be obtained by isolation as an expression product from the host cell. Recombinant proteins can be purified by conventional techniques known in the art, typically conventional chromatographic methods.

In some embodiments the lectin is specific for the TF antigen. In some embodiments the lectin is specific for O-glycans.

In some embodiments, the lectin has an IC₅₀ value of no more than 100 μg/mL, no more than 80 μg/mL, no more than 50 μg/mL, no more than 25 μg/mL, no more than 20 μg/mL, no more than 15 μg/mL or no more than 10 μg/mL against a human cancer cell line.

In some embodiments, the lectin has an IC₅₀ value of no more than 25 μg/mL against an ovary carcinoma cell line, such as human PA-1 cell line.

In some embodiments, the lectin has an IC₅₀ value of no more than 20 μg/mL against a cervical carcinoma cell line, such as the human KB cell line.

In some embodiments, the lectin has an IC₅₀ value of no more than 50 μg/mL against a colorectal carcinoma cell line, such as the human HT-29 cell line.

In some embodiments, the lectin has an IC₅₀ value of no more than 25 μg/mL against a pancreatic cancer cell line (e.g. pancreatic epitheloid carcinoma or duct epotheloid carcinoma), such as the human PANC-1 cell line.

In some embodiments, the lectin has an IC₅₀ value of no more than 10 μg/mL against a breast cancer cell line (e.g. mammary gland adenocarcinoma, breast adenocarcinoma or breast metastatic carcinoma), such as the human MDA-MB-231 cell line.

In some embodiments, the lectin has an IC₅₀ value of no more than 15 μg/mL against a bladder cancer cell line (e.g. urinary bladder carcinoma or transitional cell carcinoma), such as the human T-24 cell line.

In some embodiments, the lectin has an IC₅₀ value of no more than 15 μg/mL and 20 μg/mL against a Brain tumour cell lines such as U251MG (Glioblastoma) and IOMM-Lee (Meningioma), respectively.

IC₅₀ values for a given therapeutic agent can be determined using standard techniques as would be known by the skilled person. For example, the IC₅₀ value of a lectin for a particular type of cancer may be determined in vitro using a suitable cell line which is representative of that type of cancer. Briefly, the cell line may be treated with the lectin protein, optionally along with a control agent, which is an established anti-cancer agent. The cell cytotoxicity may be estimated in an untreated sample, the test sample and the control using processes well known to the person skilled in the art, which may involve Calcein AM Cell Viability assay or MTT assay or any other method known to the skilled person. Percentage cytotoxicity with respect to untreated cells may be calculated using formula:

Cytotoxicity=[(RFUuntreated−RFUsample)/RFUuntreated]*100

RFU: relative fluorescence units

The IC₅₀ value may be calculated using software know to the skilled person, such as Pad Prism version 4.01 software.

The lectin may be provided in a pharmaceutically acceptable form, such as a liquid (e.g in an aqueous solution or suspension, or as an oil based solution or suspension.), a solid (e.g a capsule or tablet), a lyophilized powder, a spray, cream, lotion or gel, vesicular drug delivery systems such as, but not limited to, bilosomes, liposomes, niosomes, transferosome, ethosomes, sphingosomes, pharmacosomes, multilamellar vesicles, microsphere and the like.

As used herein, an “aqueous solution” is a solution which is produced by dissolving a solid or lyophilized agent, such as a recombinant lectin having the amino acid sequence of SEQ ID NO. 1, in water or in a buffer containing water. An Aqueous solution is also formed when an agent, such as a recombinant lectin having the amino acid sequence of SEQ ID NO.1, is in liquid form and is mixed with water or a buffer containing water.

The terms “cancer”, “tumor” and “tumour”, may be used interchangeably in the present application, as would be understood by the person skilled in the art. Cancers or tumours result from abnormal cell growth. They form when the normal cells grow out of control and crowd out. Formation of tumours often affects the normal functioning of the tissue, organ or organism.

Cancer can start any place in the body and can also spread to other parts of the body.

The spread of cancer cells is referred to as metastasis. Thus the term “cancer” encompasses both primary and metastatic cancers. As used herein, the term “cancer” includes, but is not limited to, solid tumors and blood borne tumors.

The term “cancer” includes diseases of the skin, tissues, organs, bone, cartilage. Examples of cancers that may be treated by the methods and compositions of the present invention include, but are not limited to, cancer of the bile duct, bladder, bone, brain, breast, cervix, colon, oesophagus, gastrointestine (including the ileum, colon, rectum and/or anus), head, kidney, liver, lung, nasopharynx, neck, ovary, pancreas, prostate, skin, stomach, testis, tongue, thyroid, urachus, vagina & uterus. The cancer may be benign or malignant, and in any stage of malignancy.

The cancer may be a cancer of the epithelial tissues, non-epithelial tissues, the cells that make up the skin or the tissue lining the organs, cells of the immune system, connective tissue, or cells of the spinal cord or brain.

In some embodiments, the cancer may be a solid tumour.

The cancer may be a carcinoma. In some embodiments, the cancer is adenocarcinoma. The adenocarcinoma may be oesophageal, pancreatic, prostate, cervical, breast, colon or colorectal, lung, bile duct, vaginal, urachus or stomach adenocarcinoma.

In some embodiments, the cancer is squamous cell carcinoma. The squamous cell carcinoma may be skin, oral, lung, thyroid, oesophagus, vaginal, cervical, ovarian, head and/or neck, prostate or bladder squamous cell carcinoma.

In some specific embodiment the cancer may be brain tumor/cancer, which might include Glioblastoma, meningioma, astrocytoma, glioma and neuroblastoma.

It will be understood that the term “treatment” may comprise substantially curing the cancer, preventing or slowing the progression of, or reducing the severity of, the disease, preventing or reducing metastases, inhibiting tumour growth, reducing tumour mass or eliminating tumours, and/or ameliorating (either temporarily or permanently) symptoms associated with the disease. It will be appreciated that symptoms will vary depending on the type of cancer, but may include pain, reduction or loss of function, nausea and/or sickness, fever, tumour formation, immunosuppression, and/or tiredness.

The treatment may comprise administering a therapeutically effective amount of the lectin to the subject. In some embodiments, the lectin is administered at a dose of from about 0.05 mg/Kg to about 1000 mg/Kg, from about 0.1 mg/Kg to about 100 mg/Kg.

In some embodiments the treatment comprises administering the lectin to a subject such that the effective concentration of the lectin in the subject is from about 0.001 μg/mL to about 1000 μg/mL, 0.05 μg/mL to about 500 μg/mL, from 0.1 μg/mL to 200 μg/mL, from 0.15 μg/mL to 150 μg/mL.

In some embodiments, the cancer is selected from breast cancer (e.g. breast adenocarcinoma), cervical carcinoma, ovarian cancer (e.g. ovarian squamous cell carcinoma) and pancreatic cancer (e.g. pancreatic adenocarcinoma), bladder cancer (e.g urothelial carcinoma, urothelial carcinoma), Brain cancer (e.g. Glioblastoma, meningioma, astrocytoma, glioma and neuroblastoma) and the treatment comprises administering the lectin to the subject such that the effect concentration of the lectin in the subject is from 0.1 μg/mL to 200 μg/mL.

In some embodiments, the treatment comprises administering a non-cytotoxic concentration of the lectin.

Administration of the lectin may be by any suitable route, including but not limited to, injection (including intravenous (bolus or infusion), intra-arterial, intraperitoneal, subcutaneous (bolus or infusion), intraventricular, intramuscular, or subarachnoidal), oral ingestion (e.g. of a tablet, gel, lozenge or liquid), inhalation, topical, via a mucosa (such as the oral, nasal or rectal mucosa), by delivery in the form of a spray, tablet, transdermal patch, subcutaneous implant or in the form of a suppository.

The subject may be a mammalian subject. In some embodiments, the subject is human.

### Anti-Angiogenesis

Angiogenesis plays an important role in the growth and progression of cancer. Blood vessels penetrating into the tumour parenchyma provide nutrition and oxygen for multiplying cells. The control of tumour angiogenesis depends on a net balance of several activators (angiogenic factors) and inhibitors (anti-angiogenic factors), which are secreted by both tumour cells and host infiltrating cells such as macrophages and fibroblasts. The angiogenic factors induce endothelial cells to secrete proteases and plasminogen activators that degrade the vessel basement membrane, leading to cell invasion into the surrounding matrix and the formation of new vessels. Potent anti-angiogenic molecules inhibit the proliferation and migration of endothelial cells, by binding to the pro-angiogenic factors or blocking the activities of receptors on the surface of endothelial cells.

Thus, in some embodiments, the lectin is capable of inhibiting the migration and/or proliferation of cells, such as endothelial cells.

The ability of a lectin to inhibit the migration and/or proliferation of cells can be tested using standard techniques, such as those described herein.

Non-cytotoxic concentrations of recombinant lectin having the amino acid sequence of SEQ ID NO. 2 demonstrating anti-proliferative and hence anti-angiogenic effects in endothelial cells may be from 10 μg/mL to 100 μg/mL.

Evaluation of the anti-proliferative effect of the recombinant lectin was performed with Doxorubicin as a positive control. The recombinant lectin showed dose dependent inhibitory effect on serum mediated cell proliferation.

The recombinant lectins of the present invention such as having the amino acid sequence of SEQ ID NO. 2 showed dose dependent inhibitory effect on serum mediated cell proliferation. The non-cytotoxic concentrations of the recombinant lectin having the amino acid sequence of SEQ ID NO. 2 demonstrated anti-proliferative and hence anti-angiogenic effects in endothelial cells, at concentrations which ranged from about 20 μg/mL to 100 μg/mL.

The determination of the anti-angiogenic effect of a recombinant lectin having the amino acid sequence of SEQ ID NO.2 in human endothelial cells EA.Hy926 was performed with a concentration of lectin ranging from about 0.1 μg/mL to 200 μg/mL. A concentration of from 20 μg/mL to 100 μg/mL resulted in the inhibition of endothelial cell proliferation by from 15.51% to 58.53% as compared to control. The same concentration range resulted in the inhibition of endothelial cells migration by from 71.5% to 82.4% as compared to control (DMEM) after 72 hrs.

The present invention further relates to the evaluation of in-vivo anti-angiogenesis potential of recombinant lectin (such as recombinant lectin having the amino acid sequence of SEQ ID NO. 2 using Matrigel plug assay in C57BL/6 mice. The group of mice treated with recombinant lectin having the amino acid sequence of SEQ ID NO. 2 showed 23.6% reduction in haemoglobin content in homogenate of Matrigel plug, whereas the group treated with Sunitinib showed maximum reduction i.e. 59.2% in haemoglobin content in homogenate of Matrigel plug. Further neovascularization was marginally reduced in mice treated with Recombinant Lectin having the amino acid sequence of SEQ ID No 2 at 10 mg/Kg.

The present invention further relates to the evaluation of modulatory effect of recombinant lectins (such as a recombinant lectin having the amino acid sequence of SEQ ID No 2) on signaling pathways involved in pathogenesis of cancer.

The lectin may modulate one or more biomarkers selected from: MEK-1; P90RSK; STAT-3; p53; MMPs; HGF; EGF; C-kit; VEGF; VEGFR; Her-2/3; GMSCF; IL-6; IL-8; p38/MAPK; PDGF; MPO; Fol-1; CD40L; Angiopoietin-2; Osteopontin; Endoglin; P1GF; BMP-9; Endothelin-1.

The modulatory effect of a recombinant lectin having the amino acid sequence of SEQ ID NO. 2 on various signaling pathways, and on the inhibition of MAPK/EGFR/Ras/Raf, CCR5, IL-4/STATE, NF-KB, PI3K/AKT/FOXO3, PKC/Ca2+ and TNF-alpha/JNK pathways which play crucial role in cancer, is demonstrated.

In some embodiments, the lectin modulates one or more biomarkers or signaling pathways selected from MAPK/EGFR/Ras/Raf; ADBR1; CCR5; NF-KB; PI3K/AKT/FOXO3; and PKC/CA2+.

The inhibiting concentration range of the recombinant lectin for the MAPK/EGFR/Ras/Raf and ADBR1 pathways is between 0.158 μg/mL to 50 μg/mL with an effective inhibition from 2% to 48% and from 26% to 49%, respectively for the two pathways.

The inhibiting concentration range of the recombinant lectin for the NF-KB, TNF-alpha/JNK and PI3K/AKT/FOXO3 pathways is between 0.5 μg/mL to 50 μg/mL with an effective inhibition from 3% to 13%, from 12% to 45% and from 2% to 73%, respectively for the 3 pathways.

The inhibiting concentration range of the recombinant lectin for the CCR5 pathway is in the range of 0.058 μg/mL to 50 μg/mL with effective inhibition of 21%-70%.

The inhibiting concentration range of the recombinant lectin for the PKC/Ca2+ pathway in the range of 0.00158 μg/mL to 0.5 μg/mL with an effective inhibition from 5% to 19%.

In some embodiments, the lectin modulates VEGF levels. It will be understood that, as used herein, the term “modulates” refers to the ability of an agent to increase or decrease the expression or activity level of a biomarker or signalling pathway, compared to normal levels (i.e. in untreated cells). In some embodiments, the lectin increases expression of VEGF in cells, such as cancer cells.

Vascular epidermal growth factor (VEGF) is an important angiogenic molecule associated with neovascularization and a key regulator of vascular endothelial cell regeneration. A reduction in VEGF levels is usually associated with anti-angiogenic properties. However, the activity of some anti-cancer agents, such as proteasome inhibitor (PSI) which have been shown to exert significant antitumor effects against C-26 colon carcinoma, has been associated with the upregulation of VEGF, at both the level of mRNA expression and protein production. It has been suggested that higher VEGF production may render endothelial cells susceptible to the proapoptotic activity of PSI and is associated with inhibition of tumor growth.

Surprisingly, the present inventors have found that the treatment of cancer cells with a lectin in accordance with the present invention resulted in an increase in VEGF levels, as compared to untreated cells. Without being bound by theory, it is thought that the lectins of the present invention may exert their anti-cancer effect in a similar way to PSI, by increasing the susceptibility of endothelial cells to the pro-apoptitic activity of the lectin, thereby exerting an anti-angiogenic effect.

Thus, in some embodiments, the lectin further induces apoptosis of cancer cells.

The anti-angiogenic effect of the lectin may be determined by a reduction of tumor mass or volume, by the percentage inhibition of tumour growth (% TGI) or by the disappearance of tumours. In some embodiments, the anti-angiogenic effect of the lectin may be determined by an increase in the time required for a tumour to reach a predetermined mass or volume, as compared to an untreated control.

In some embodiments, the lectin inhibits (or is capable of inhibiting) tumour growth by at least 20%, 30%, 35%, 40%, 45%, 50%, 55%, or at least 60%. The % tumour growth inhibition (% TGI) can be determined using methods described herein.

In some embodiments, the lectin effects (or is capable of effecting) a tumour growth delay of at least 2, 3, 4, 5, 6, 7, 8, 10, 12 or at least 14 days. The Tumour Growth Delay (TGD) can be determined using methods described herein.

According to a further aspect of the present invention, there is provided a method of treating cancer in a subject, the method comprising administering a lectin to the subject, wherein the lectin effects treatment of the cancer by inhibiting angiogenesis.

The present invention further relates to a method of preventing angiogenesis in a tumor in a subject, using a lectin in accordance with the present invention.

In some embodiments, the method of preventing angiogenesis in the tumor comprises using a non-cytotoxic concentration of the lectin, such as a recombinant lectin having the amino acid sequence of SEQ ID NO. 1 or its homologous sequence. The non-cytotoxic concentration of the lectin may be from about 0.1 μs/mL to about 200 μg/mL. The method may comprise contacting a solution of the lectin (e.g. solution of recombinant lectin having the amino acid sequence of SEQ ID NO.1 or its homologous sequence, at a concentration of about 0.1 μg/mL to about 200 μg/mL) with tumor cells.

In some embodiments, the present invention relates to effecting anti-angiogenesis in tumor cells using from about 0.1 mg/Kg to 100 mg/Kg body weight of a lectin, such as a recombinant lectin comprising the amino acid sequence of SEQ ID NO.1 or its homologous sequence.

As used herein, references to mg/Kg of body weight refer to mammalian body weight, such as human body weight.

According to the aspect of the solution of recombinant lectin having amino acid sequence of SEQ ID 1 or its homologous sequence with concentration of about 0.1 μg/mL to about 200 μg/mL, is contacted with the tumor cells of mammalian body with amount from about 0.1 mg/Kg to 100 mg/Kg of the mammalian body weight for effective anti-angiogenesis effect in the tumor cells.

### Apoptosis

According to yet another aspect of the invention there is provided a lectin for use in a method of treatment of cancer by inducing apoptosis.

The invention provides a recombinant lectin for use in a method of treatment of cancer by inducing apoptosis in the cancer cell comprising administration of therapeutically effective amount of a recombinant lectin protein. According to this aspect the lectin induces early and late stage of apoptosis in the cancer cells.

The present invention further relates to a method of inducing apoptosis in a tumor in a subject, using a recombinant lectin in accordance with the present invention.

The present invention also relates to the evaluation of in vitro apoptotic effect of a lectin (such as a recombinant lectin having the amino acid sequence of SEQ ID NO. 1 or SEQ ID NO. 2) on a cancer cell line, for example on a breast cancer cell line and/or a pancreatic cancer cell line. The in vitro apoptotic effect of lectins may be determined using standard assay techniques which are known to the skilled person.

The evaluation of the in vitro apoptotic effect of a recombinant lectin having the amino acid sequence of SEQ ID NO. 2 on MDA-MB-231 as a breast cancer cell line and PANC-1 cell line as a pancreatic cancer cell line was carried out using a JC-1 assay. The evaluation was performed using a concentration of recombinant lectin of about 2.5 μg/mL-80 μg/mL, with Doxorubicin as a positive control.

The recombinant lectin having the amino acid sequence of SEQ ID NO. 2 led to a significant depolarization of mitochondrial membrane in the range of 9.5%-51.7% for PANC-1 cell line and 19.8%-54.1% for MDA-MB-231 cell line.

The evaluation of in vitro apoptotic effect of SEQ ID NO. 2 on MDA-MB-231 and PANC-1 cell lines using Annexin-V staining indicated the recombinant lection had early and high late apoptotic effect on the cancer cell lines as compared to the standard (doxorubicin) which showed only late apoptotic effect. Further the cell cycle analysis indicated enhancement of apoptotic cell population on treatment with SEQ ID NO. 2.

The present invention further relates to the evaluation of modulatory effect of recombinant lectins (such as a recombinant lectin having the amino acid sequence of SEQ ID NO.2) on signaling pathways involved in pathogenesis of cancer.

SEQ ID NO. 2 induced apoptosis by modulation of one or more biomarkers selected from: MEK-1; P90RSK; STAT-3; p53; C-kit; IL-6; IL-8; p38/MAPK; MPO; Fol-1; CD40L; ATF-2, ERK1/2; JNK; TNFR; Galectin-3; Kalikrein-5 and TNF-α.

The modulatory effect of a recombinant lectin having the amino acid sequence of SEQ ID NO. 2 on various signaling pathways, and on the inhibition of MAPK/EGFR/Ras/Raf, CCR5, IL-4/STATE, NF-KB, PI3K/AKT/FOXO3, PKC/Ca2+ and TNF-alpha/JNK pathways which play crucial role in cancer, is demonstrated.

In some embodiments SEQ ID NO. 2 modulates one or more biomarkers or signaling pathways selected from IL-4/STAT6; NF-KB; PI3K/AKT/FOXO3; and TNF-α/JNK.

The inhibiting concentration range of the recombinant lectin for the NF-KB, PI3K/AKT/FOXO3 and TNF-alpha/JNK pathways is between 0.5 μg/mL to 50 μg/mL with an effective inhibition from 3% to 13%, from 2% to 73% and from 12% to 45%, respectively for the 3 pathways.

The inhibiting concentration range of the recombinant lectin for the IL-4/STAT6 pathway is in the range of 0.0158 μg/mL to 0.5 μg/mL with an effective inhibition from 16% to 28%.

In some embodiments, the method of inducing apoptosis in the tumor comprises using a non-cytotoxic concentration of the lectin, such as a recombinant lectin having the amino acid sequence of SEQ ID NO. 1 or homologous sequence thereof. The non-cytotoxic concentration of the lectin may be from about 0.1 μg/mL to about 200 μg/mL. The method may comprise contacting the recombinant lectin or the solution of recombinant lectin having the amino acid sequence of SEQ ID NO.1 or its homologous sequence, at a concentration of about 0.1 μg/mL to about 200 μg/mL with tumor cells.

In some embodiments the present invention relates to effecting apoptosis in tumor cells using from about 0.1 mg/Kg to 100 mg/Kg body weight of a recombinant lectin, such as a recombinant lectin comprising the amino acid sequence of SEQ ID NO.1 or its homologous sequence.

The treatment of the cancer or the effective apoptosis of the tumor cells may be determined by a reduction in tumour volume or by the disappearance of one or more tumours.

In some embodiments, the recombinant lectin comprises or consists of the amino acid sequence of SEQ ID No 1 or SEQ ID No 2 or SEQ ID No 3, or SEQ ID No 4.

### Composition:

The method of treatment of cancer, the method of preventing angiogenesis, or the method of inducing apoptosis may comprise contacting the tumour with a composition comprising the recombinant lectin. For example, a solution of recombinant lectin having amino acid sequence of SEQ ID NO.1 or its homologous sequence may be contacted with the tumour cells. The concentration of the recombinant lectin in the composition may be from about 0.001 μg/mL to about 1000 μg/mL, about 0.05 μg/mL to about 500 μg/mL, from about 0.1 μg/mL to about 200 μg/mL, or from about 0.15 μg/mL to about 150 μg/mL.

The method of treatment of cancer, the method of preventing angiogenesis or the method of inducing apoptosis may comprise administering the recombinant lectin in an amount of from about 0.05 mg/Kg to about 1000 mg/Kg, or from about 0.1 mg/Kg to about 100 mg/Kg of a mammalian body weight.

According to a further aspect a method of treatment of adenocarcinoma or squamous carcinoma or brain cancer by preventing angiogenesis and/or by inducing apoptosis in tumour cells, wherein the method comprises contacting the tumour cells with a solution of a recombinant lectin, such as a recombinant lectin having the amino acid sequence of SEQ ID NO. 1 or its homologous sequence. The concentration of the solution of the recombinant lectin (e.g. SEQ ID NO.1, or SEQ ID NO. 2) may be is from about 0.1 μg/mL to 200 μg/mL.

The present invention further relates to the evaluation of anti-tumor potential of recombinant lectins, such as a recombinant lectin having the amino acid sequence of SEQ ID NO. 2.

The anti-tumor potential of a recombinant lectin having the amino acid sequence of SEQ ID NO. 2 was evaluated using a PA-1 (ovary teratocarcinoma) xenograft model. Nude mice treated with recombinant Lectin showed significant decrease in tumor size upon treatment.

The anti-tumor potential of the recombinant lectin was also evaluated using a KB (cervical carcinoma) xenograft model. The percentage tumour growth inhibition was comparable to the standard (Doxorubicin).

The anti-tumor potential of the recombinant lectin was further evaluated using a HT-29 (colorectal adenocarcinoma) xenograft model. Animals treated with the recombinant lectin at 20 mg/Kg and 30 mg/Kg body weight (daily) showed a significant decrease in tumor volume in comparison with the vehicle control group. Animals were further treated with the recombinant lectin using a T24 (urinary bladder/transitional cell carcinoma) Xenograft Model. The Recombinant Lectin showed appreciable anti-tumor potential which was comparable to a Doxorubicin-treated group in the T24 xenograft model.

The anti-tumor potential of the recombinant lectin was further evaluated using breast cancer cell lines (MCF-7 and MDA-MB-231). The percentage tumour growth inhibition was comparable to the standard (Doxorubicin) in both the cases.

Further PANC-1 (Pancreatic/duct epithelioid carcinoma) cell lines were also treated with the recombinant lectin The Recombinant Lectin showed appreciable anti-tumor potential as compared to a Gemcitabine-treated cell lines.

The present invention further relates to a recombinant lectin (e.g. a recombinant lectin having the amino acid sequence of SEQ ID NO.2) for use as an angiogenesis inhibitor or apoptosis inducer, thereby inhibiting cancer cell metastases and/or causing programmed cell death.

It will be appreciated that any of the embodiments described herein may be combined with each other and with any aspect of the invention, unless otherwise stated.

The present set of examples demonstrate the best mode of performance and do not restrict the scope of the invention in any manner.

### EXAMPLES

### Example 1: Anti-Cancer Potential Recombinant Lectin Having Amino Acid Sequence of SEQ ID No. 2

The purified recombinant lectin of SEQ ID No 2 was studied for its anti-cancer potential in different cell lines. It has shown the cytotoxic activity in the 10 different cancer cell lines. The brief method of the assay was as follows:
1. The specific number of cancer/normal cells were plated in 96-well tissue culture plate
2. After overnight incubation the cells were treated with respective test item for predetermined time-interval (48 hrs-72 hrs)
3. The cytotoxicity/anti-proliferative activity of the test items was estimated by chemiluminescence/fluorescence/colorimetric detection method
4. The percentage cytotoxicity was calculated using the statistical tool.

The recombinant lectin having amino acid sequence of SEQ ID No 2 showed cytotoxic effect on all 10 cancer cell lines tested and did not show cytotoxic effect on the normal cells (PBMCs). It showed better effect in MDA-MB-231 (triple negative breast adenocarcinoma cells) compared to MCF-7 (Breast adenocarcinoma). The results are summarized in table below.

Summary of cell based in vitro cytotoxicity/anti-proliferation assay

| | | | % Cytotoxicity | |
|---|---|---|---|---|
| | | | SEQ ID | Doxorubicin/ |
| S. No. | Cell line | Type of Cancer | NO 2 | 5-FU |
| 1 | AGS | Stomach | 65.3 | 81.0 |
| 2 | HT-29 | Colon | 54.9 | 75.0 |
| 3 | PA-1 | Ovary | 72.0 | 82.3 |
| 4 | KB | Cervical | 64.0 | 92.0 |
| 5 | MCF-7 | Breast | 28.0 | 64.9 |
| 6 | MDA-MB-231 | Breast | 60.9 | 50.2 |
| 7 | MDA-MB-453 | Breast | 46.6 | 71.4 |
| 8 | MIAPaCa-2 | Pancreas | 47.5 | 63.9 |
| 9 | PANC-1 | Pancreas | 60.8 | 65.1 |
| 10 | T-24 | Bladder | 93.1 | 54.5 |
| 11 | PBMC | Peripheral blood | Nil | 32.0 |
| | | mononuclear cells | | |

### Example 2: In Vivo (Xenograft) Efficacy Studies

As per above data, the recombinant lectin of SEQ ID No 2 exhibited cytotoxic anti-proliferation effect on various cancer cell lines in in vitro assays. The efficacy of the recombinant lectin having amino acid sequence of SEQ ID No 2 as antitumor agent was assessed in respective Xenograft in immunocompromised mice models in vivo. The Xenograft models used were HT-29, KB, PA-1, MCF-7, PANC-1, T24 and MDA-MB-231. The basic study design for the Xenograft study was as follows:
1. Cell maintenance and cell suspension preparation
2. Aseptic injection of tumor cell suspension in the donor animal
3. Randomization of animals in respective groups
4. IP dosing of test items in predetermined doses
5. IP administration of standard
6. After completion of dosing period.
7. The Tumor volume recording weekly twice while body weight and clinical signs daily.

The results of individual Xenograft studies are summarized in table below.

Xenograft studies

| | | |
|---|---|---|
| | Tumor Volume (mm³) | Maximum Tumor Growth |
| Group & Treatment | (Mean ± SEM) | Inhibition (%) |

HT-29 (Colorectal cancer)

| | | |
|---|---|---|
| Vehicle Control | 3675 ± 937 | 0 |
| SEQ ID NO 2 | 1702 ± 274 | 56.62 |
| 5-Fluorourasil | 1362 ± 155 | 62.94 |

KB (Cervical cancer)

| | | |
|---|---|---|
| Vehicle Control | 1189 ± 202 | 0 |
| SEQ ID NO 2 | 782 ± 92 | 60.49 |
| Doxorubicin | 616 ± 118 | 64.60 |

PA-1 (Ovary teratocarcinoma)

| | | |
|---|---|---|
| Vehicle Control | 2360 ± 278 | 0 |
| SEQ ID NO 2 | 1050 ± 132 | 62.15 |
| Doxorubicin | 631 ± 87 | 84.48 |

MCF-7 (Breast cancer)

| | | |
|---|---|---|
| Vehicle Control | 4204 ± 897 | 0 |
| SEQ ID NO 2 | 1256 ± 132 | 69.04 |
| Doxorubicin | 771 ± 65 | 65.23 |

T24 (Urinary bladder/transitional cell carcinoma)

| | | |
|---|---|---|
| Vehicle Control | 843.1 ± 51.67 | 0 |
| SEQ ID NO 2 | 551.06 ± 10.02 | 36.52 |
| Doxorubicin | 366.32 ± 34.28 | 59.07 |

PANC-1 (Pancreas/duct epithelioid carcinoma)

| | | |
|---|---|---|
| Vehicle Control | 1078.74 ± 214.93 | 0 |
| SEQ ID NO 2 | 721.17 ± 194.63 | 39.15 |
| Gemcitabine | 503.93 ± 151.27 | 61.49 |

MDA-MB-231 (Mammary gland/breast adenocarcinoma)

| | | |
|---|---|---|
| Vehicle Control | 952.60 ± 53.61 | 0 |
| SEQ ID NO 2 | 834.80 ± 126.95 | 35.21 |
| Doxorubicin | 808.47 ± 178.59 | 42.12 |

The effect of recombinant lectin having amino acid sequence of SEQ ID NO 2 on tumour volume and Tumour growth inhibition depicted in above table recombinant lectin having amino acid sequence of SEQ ID NO 2 showed strong anti-cancer activity in immunocompromised mice model in various cancers.

### Example 3: Evaluation of Modulatory Effect of Recombinant Lectin on Signaling Pathways Involved in Pathogenesis of Cancer

The mechanism of action of the recombinant lectin having the amino acid sequence of SEQ ID NO.2 is studied by determining its effect on the modulation of key signaling pathways involved in the pathogenesis of cancer. The study was performed using SelectScreen® Cell-based Pathway Profiling Services at Life Technologies, USA. Modulatory effect of Lectin having the amino acid sequence of SEQ ID NO. 2 on various cell signaling pathways was investigated in cell lines overexpressing specific markers using GeneBLAzer Beta-lactamase (bla) Reporter Technology and Tango platforms. The cell lines tested were MDA-MB-231 (Human Breast cancer), KB (Human Cervical cancer), PA-1 (Human ovarian cancer), PANC-1 (Human pancreatic cancer), HT-29 (Human colorectal cancer), T-24 (Human Bladder cancer)

10 mg of recombinant lectin having the amino acid sequence of SEQ ID NO.2 was dissolved in 200 μL of TBS buffer (25 mM, pH 8.0) to obtain stock solution of 50 mg/mL. The stock solutions were diluted in Serum Free Medium (STM) to achieve final concentrations in cells ranging from 0.00158 μg/mL-50 μg/mL. Cells (32 μL) were diluted in Assay Media to appropriate cell density and were added to the assay plate. Cells were incubated at 37° C./5% CO2 for 24 h. 40 nL of 1000×sample and 4 μL of assay medium was added to the cells in the assay plate and incubated for 30 minutes at 37° C./5% CO2 in a humidified incubator. Then, 4 μL of the 10×EC80 concentration of activator was added to all wells containing sample to bring the final assay volume to 40 μL. The assay plate was incubated for 16 hours at 37° C./5% CO2 in a humidified incubator. Further, 8 μL of the Substrate Loading Solution (LiveBLAzer™—FRET B/G) was added to the assay plate. The assay plate was incubated for 2 hours at room temperature, in the dark. The assay plate was read on a fluorescence plate reader (Tecan Safire2). Fluorescence emission values at 460 nm and 530 nm were obtained using a standard florescence plate reader and % modulation was determined.

% Modulation=[(A−B)/A]*100

Where, A=Fluorescence reading in Control (untreated cells)
B=Fluorescence reading in TI treated cells

### Results

The lectin resulted in the inhibition of signalling pathways as shown in Table below:

| | | Effect |
|---|---|---|
| Pathway/Marker | Concentration range | (wrt control) |
| MAPK/EGFR/ | 0.158 μg/mL-50 μg/mL | 2%-48% inhibition |
| Ras/Raf | | |
| ADBR1 | 0.158 μg/mL-50 μg/mL | 26%-49% inhibition |
| CCR5 | 0.058 μg/mL-50 μg/mL | 21%-70% inhibition |
| IL-4/STAT6 | 0.0158 μg/mL-0.5 μg/mL | 16%-28% inhibition |
| NF-KB | 0.5 μg/mL-50 μg/mL | 3%-13% inhibition |
| PI3K/AKT/FOXO3 | 0.5 μg/mL-50 μg/mL | 2%-73% inhibition |
| PKC/Ca2+ | 0.00158 μg/mL-0.158 μg/mL | 5%-19% inhibition |
| TNF-alpha/JNK | 0.5 μg/mL-50 μg/mL | 12%-45% inhibition |

The effect of modulation of mechanistic biomarkers by recombinant lectin having the amino acid sequence of SEQ ID NO.2 is summarized as below:
increased levels of HGF, which binds to its receptor C-Met, and then acts on MAPK,
activated ATF-2 which is a tumor suppressor and acts via inducing apoptosis,
increased expression of c-kit, which is receptor for SCF, this acts downstream on MAPK,
increased the expression of JNK and inhibits p53, which activates AFT-2 resulting in apoptosis,
stimulated VEGF, VEGF-A and VEGFR2 expression, which activates Ras-Raf pathway,
inhibited expression of EGF, which binds to EGFR and exerts anticancer effect via Ras-Raf pathway or PKC,
stimulated Her-2, which activates Ras-Raf-MEK1-MAPK-ERK activation,
increased ERK and MEK-1 levels, which activated p90RSK and MMPs expression. ERK also acts via stat-3 and NF-KB,
stimulated GMCSF and IL-6 expression, which act via JAK-STAT pathway and activates NF-KB,
activated IL-4R expression, which connects to JAK-STAT pathway. IL-4R also acts via PI3K-Akt arm,
increased IL-8 levels, which acts via PI-3K-Akt-FOX3 arm. Akt also acts via Stat-3-NF-KB arm, where SEQ ID NO. 2 exerted an increase in Stat-3,
increased expression of Leptin, which binds to its receptor Leptin-R and acts on ERK pathway,
increased levels of cytokines (GMCSF, IL-6 and IL-8) may also interact with the immune system in tumor microenvironment to mount anticancer immunoprotective effect,
activated of TNF-α which connects to JNK pathway,
increased levels of endoglin-1 expression, which is a receptor for TGF-β. Endoglin-1 acts via Smad and activates ATF-2 expression,
activation of PDGF-BB PDGF exerts antitumor effects via suppressing angiogenesis,
activation and TGF-alpha regulates cancer cell growth through autocrine and paracrine pathways,
activation of Angiopoietin-1 & Angiopoietin-2 enhances infiltration by TIE2-expressing macrophage, which exhibit tumor suppressive functions. Binding to Tie-2 regulates PI3k pathway,
activation of Kallikrien-3 & Kallikrien-5 that acts as act a tumor suppressor, via induction of apoptosis,
activation of TRAIL & TRAILR2 causes apoptosis primarily in tumor cells, by binding to certain death receptors,
activation of Osteopontin (PON) which binds to receptors αVβ3 and CD44 and acts via Akt pathway,
inhibited Galectin-1 which either acts via Ras-Raf pathway or PI3k-Akt pathway. It also inhibited Galectin-3, which acts via PI3k-AKT pathway, increased Fol-1 expression, which acts via Smad and activates ATF-2 expression,
inhibited CD40L, which binds to CD40 and inhibits MAPK.

### Example 4: Evaluation of In Vitro Apoptotic Effect of Lectin

To evaluate the in vitro apoptotic effect of recombinant lectin having the amino acid sequence of SEQ ID NO.2 in MDA-MB-231 (breast cancer) and PANG-1 (pancreatic cancer) cell line by JC-1 assay, an increase in mitochondrial membrane depolarization was used as a marker of apoptosis. The extent of mitochondrial membrane depolarization was assessed in MDA-MB-231 and PANG-1 cells after 16h of treatment with recombinant lectin having amino acid sequence of SEQ ID NO. 2 using JC-1 dye based method. The cell lines MDA-MB-231 (Human breast adenocarcinoma) and PANC-1 (Human pancreatic epitheloid carcinoma) were procured from National Centre for Cell Science, Pune (India). The cell lines were grown in Dulbecco's Modified Eagle Medium (DMEM)+10% heat inactivated FBS at 37° C. (95% humidity and 5% CO2). The cell line was subcultured by trypsinization followed by splitting the cell suspension into fresh flasks and supplementing with fresh culture medium. Recombinant lectin having the amino acid sequence of SEQ ID NO. 2 was diluted in serum free medium. Doxorubicin was used as positive control and stock solution was prepared in dimethyl sulfoxide (DMSO). The MDA-MB-231 and PANC-1 cells were trypsinized, counted and plated in wells of flat bottom 96-well plate (dark walled plate) at the density corresponding to 10×10³ cells/well/180 μl DMEM with 10% FBS. The cells were then incubated overnight under growth conditions to allow the cell recovery and exponential growth. Cells were treated with the recombinant lectin of SEQ ID NO. 2 (20 μl stock solution) to achieve final concentrations of 2.5 μg/mL, 5 μg/mL, 10 μg/mL, 20 μg/mL, 40 μg/mL and 80 μg/mL. Similarly cells were treated with doxorubicin to achieve final concentrations of 0.1 μM, 1 μM, 10 μM, 25 μM and 50 μM. Following respective treatments the cells in the above plate were incubated for 16h in CO2 incubator at 37° C., 5% CO2 and 95% humidity.

After 16h of incubation, the supernatants were discarded and 100 μL of JC1-dye solution (prepared by diluting 1 mM DMSO stock in to 10 μM in 1×PBS) was added to each well. The cells were then incubated with the dye in CO2 incubator at 37° C. for 15 min. After 15 min of incubation, the supernatant was removed and the cells were washed twice with 1×PBS and then 100 μL of 1×PBS was added to each well. Red fluorescence (excitation 550 nm, emission 600 nm) and green fluorescence (excitation 485 nm, emission 535 nm) were measured using Biotek Synergy FIT plate reader. The mitochondrial membrane potential (ATM) was calculated as the ratio of intensity of red fluorescence to intensity of green fluorescence described as follows:

ΔΨM=Intensity of red fluorescence/Intensity of green fluorescence

The percentage decrease of Red fluorescence/Green fluorescence corresponding to each treatment was calculated using the following formula:

% Decrease=[(R−X)/R]*100

Where X=Δψm corresponding to treated cells
R=ΔΨM corresponding to control wells.

It was observed that the recombinant lectin led to the depolarization of mitochondrial membrane in the range of 9.5%-51.7% for the PANC-1 cell line and 19.8%-54.1% for the MDA-MB-231 cell line (Table 1 and 2).

TABLE 1
Percentage Decrease in mitochondrial membrane
potential (MMP) for MDA-MB-231 cell line.

| | | | Average | Average | | Percentage |
|---|---|---|---|---|---|---|
| | | | Red | Green | Red/ | decrease |
| | | | fluores- | fluores- | Green | in MMP |
| Sample | Concentration | | cence | cence | ratio | (%) |
| Untreated | — | | 1348.5 | 565.3 | 2.39 | 0 |
| Doxorubicin | 0.1 | μM | 465.3 | 473.0 | 0.98 | 58.8 |
| (μM) | 1 | μM | 343.3 | 345.5 | 0.99 | 58.3 |
| | 10 | μM | 580.7 | 534.0 | 1.09 | 54.4 |
| | 25 | μM | 376.7 | 336.3 | 1.12 | 53.0 |
| | 50 | μM | 663.7 | 318.0 | 2.09 | 12.5 |
| SEQ ID | 2.5 | μg/mL | 769.7 | 703.0 | 1.09 | 54.1 |
| NO. 2 | 5 | μg/mL | 709.3 | 560.0 | 1.27 | 46.9 |
| | 10 | μg/mL | 1098.3 | 596.3 | 1.84 | 22.8 |
| | 20 | μg/mL | 929.3 | 497.3 | 1.87 | 21.7 |
| | 40 | μg/mL | 1009.0 | 535.7 | 1.88 | 21.0 |
| | 80 | μg/mL | 957.3 | 500.3 | 1.91 | 19.8 |

TABLE 2
decrease in mitochondrial membrane potential
(MMP) for PANC-1 cell line

| | | | Average | Average | | Percentage |
|---|---|---|---|---|---|---|
| | | | Red | Green | Red/ | decrease |
| | | | fluores- | fluores- | Green | in MMP |
| Sample | Concentration | | cence | cence | ratio | (%) |
| Untreated | — | | 517.7 | 247.7 | 2.09 | 0 |
| Doxorubicin | 0.1 | μM | 653.7 | 258.5 | 2.53 | −21.0 |
| (μM) | 1 | μM | 555.3 | 282.3 | 1.97 | 5.9 |
| | 10 | μM | 350.0 | 208.3 | 1.68 | 19.6 |
| | 25 | μM | 283.0 | 186.3 | 1.52 | 27.3 |
| | 50 | μM | 192.5 | 161.7 | 1.19 | 43.0 |
| SEQ ID | 2.5 | μg/mL | 595.7 | 315.0 | 1.89 | 9.5 |
| NO. 2 | 5 | μg/mL | 452.3 | 293.3 | 1.54 | 26.2 |
| | 10 | μg/mL | 301.7 | 271.7 | 1.11 | 46.9 |
| | 20 | μg/mL | 265.7 | 234.3 | 1.13 | 45.8 |
| | 40 | μg/mL | 266.0 | 263.3 | 1.01 | 51.7 |
| | 80 | μg/mL | 272.0 | 257.0 | 1.06 | 49.4 |

### Example 5: Evaluation of Pro-Apoptotic Effect of Lectin in Human Breast Cancer Cell Line (MDA-MB-231) and Human Pancreatic Cancer Cell Line (PANC-1) by Annexin-V Staining and Cell Cycle Analysis

Cell lines MDA-MB-231 (Human breast adenocarcinoma) and PANC-1 (Human pancreatic epitheloid carcinoma) were procured from National Centre for Cell Science, Pune (India). The cell lines were maintained in DMEM+10% FBS (heat inactivated) at 37° C. with 5% CO2, and 95% humidity. Antibiotics Penicillin (100 U/mL) and Streptomycin (100 μg/mL) were added to the medium. The cell lines were subcultured by trypsinization, followed by splitting the cell suspension into fresh flasks and supplementing with fresh culture medium.

The stock solution of recombinant lectin having the amino acid sequence of SEQ ID NO. 2 was diluted in Serum Free Medium (SFM) at different concentrations corresponding to 10 fold high concentrations (weight by volume) of final effective concentration. Doxorubicin was used as positive control and stock solution was prepared in DMSO.

### Annexin Staining:

Cells were counted using hemocytometer and plated in culture plates at the density of 0.4×10⁶ cells/well in DMEM+10% FBS in 6-well plates. Cells were incubated overnight to allow the cell recovery and exponential growth. Following overnight incubation, cells were treated with Lectin having the amino acid sequence of SEQ ID NO. 2 in DMEM+0% FBS at concentrations ranging from 2.5 μg/mL to 80 μg/mL. Untreated cells were included as Control group for sample. Cells treated with Doxorubicin were included as Positive Control group. DMSO treated cells were included as control group for Doxorubicin. After treatment, cells were incubated for a time period of 24 h.

Following incubation, the pro-apoptotic effect was estimated using Annexin assay kit as follows: Annexin reagent contains Annexin-V+7-AAD stain, which differentially labels apoptotic cells in different phases. Four populations of cells can be determined from the flow cytometric plots as follows:
a) Upper Left (UL)—7-AAD(+)/Annexin(−) or Necrotic cells
b) Upper Right (UR)—7-AAD(+)/Annexin(+) or Late apoptotic cells
c) Lower Left (LL)—7-AAD(−)/Annexin(−) or Viable/non-apoptotic cells
d) Lower Right (LR)—7-AAD(−)/Annexin(+) or Early apoptotic cells

Cells were gently harvested into pre-labeled sterile centrifuge tubes and centrifuged at 300×g for 5-7 min. Supernatant were discarded and the pellet was resuspended in 200 μl of fresh culture medium.

100 μl of cell suspension was transferred into pre-labeled sterile centrifuge tubes.

100 μl of Annexin-V reagent was added to each tube and incubated for 30 min at RT in dark.

Cells stained for Annexin-V were then transferred into 96-well plates and acquired on flow cytometer (Guava technologies). Percentage of cells in early apoptotic, late apoptotic and necrotic phase were determined.

Fold increase in apoptotic cells (treated with Test Items) was determined as compared to Control (untreated cells).

### Cell Cycle Analysis

Cells were counted using hemocytometer and plated in culture plates at density of 0.5×10⁶ cells/well in DMEM+10% FBS in 6-well plates. Cells were incubated overnight to allow the cell recovery and exponential growth. Following overnight incubation, cells were sera starved in DMEM+1% FBS for 4 h. After 4 h, cells were treated with Test Items in DMEM+0% FBS at concentrations ranging from 2.5 μg/mL-80 μg/mL. Untreated cells were included as Control group for test item. Cells treated with Doxorubicin were included as Positive Control group. DMSO treated cells were included as control group for Doxorubicin. After treatment, cells were incubated for a time period of 24 h. Following incubation, the pro-apoptotic effect by cell cycle was determined as follows: Cell cycle reagent contains PI stain, which stains DNA of cells in different phases of cell cycle; Sub(G0/G1), G1, S, G2 and M. Cells in Sub(G0/G1) phase correspond to apoptotic cells.

### Harvesting and Fixation

Cells were gently harvested into pre-labeled centrifuge tubes and centrifuged at 450 g for 5 min, RT (low brake). The supernatants were removed carefully (not to touch the pellet) and discarded. 1 mL of 1×PBS was added to the pellet and resuspended gently to make homogenous suspension. Cells were centrifuged at 450 g for 5 min, RT (low brake) (washing step). The supernatant was carefully removed leaving behind approx 100 μL of PBS. Cells were resuspended gently yet thoroughly in residual PBS. Ice-cold 70% ethanol (300 μLI) was added drop-wise into cells in each tube while vortexing at low speed (fixation step). Cells were stored at 4° C. for 24 h prior to staining.

### Staining

Ethanol fixed cells were centrifuged at 450 g for 5 min, RT (low brake). The supernatant was carefully removed (not to touch the pellet) and discarded. (The pellet might not be visible but makes a thin film on surface of tube). 1 mL of 1×PBS was added into pellet and resuspended gently. Cells were incubated for 1 min at RT. Cells were centrifuged at 450 g for 5 min, RT (low brake) (washing step). The supernatant was removed carefully leaving behind approx 20 μL-50 μL of PBS. 200 μL of Cell Cycle reagent was added into each tube.

Cells were resuspended gently, mixed and were incubated for 30 min, RT, Dark. The stained samples were transferred into 96-well plates and acquired on flow cytometer (Guava technologies). Percentage of cells in Sub (G0/G1) phase were determined. Fold increase in apoptotic cells (treated with Test Items) was determined as compared to Control (untreated cells).

### Results

The results (as depicted in Table 3 to 10 below) demonstrate that the lectin induced an increase in late apoptotic and necrotic cells. Additionally, enhancement of apoptotic (SubG0/G1) cell population was also observed upon treatment of cells with the lectin.

TABLE 3
Apoptotic effect of Lectin having the amino acid sequence
of SEQ ID NO. 2 in MDA-MB-231 cells by Annexin-V staining

| | | | % Population of cells | | | |
|---|---|---|---|---|---|---|
| | | | | LR | UR | |
| | | | LL | Annex+ | Annex+ | UL |
| | | | Annex− | 7AAD− | 7AAD+ | Annex− |
| | | | 7AAD− | Early | Late | 7AAD+ |
| Sample (in MDA-MB-231) | | | Viable | apoptotic | apoptotic | Necrotic |
| Control | | | 98.1 | 0.5 | 0.6 | 0.7 |
| SEQ ID | 2.5 | μg/mL | 75.7 | 7.9 | 13.2 | 3.2 |
| NO. 2 | 5 | μg/mL | 72.3 | 7.0 | 8.2 | 12.5 |
| | 10 | μg/mL | 64.1 | 5.8 | 9.5 | 20.6 |
| | 20 | μg/mL | 58.5 | 2.9 | 5.9 | 32.7 |
| | 80 | μg/mL | 61.0 | 5.3 | 7.7 | 26.0 |
| Doxoru- | 10 | μM | 0.7 | 6.8 | 92.5 | 0.0 |
| bicin | 100 | μM | 0.0 | 0.0 | 100.0 | 0.0 |

TABLE 4
Apoptotic effect of Lectin having the amino acid sequence
of SEQ ID NO. 2 in PANC-1 cells by Annexin-V staining

| | | | % Population of cells | | | |
|---|---|---|---|---|---|---|
| | | | | LR | UR | |
| | | | LL | Annex+ | Annex+ | UL |
| | | | Annex− | 7AAD− | 7AAD+ | Annex− |
| | | | 7AAD− | Early | Late | 7 AAD+ |
| Sample (in PANC-1) | | | Viable | apoptotic | apoptotic | Necrotic |
| Control | | | 89.2 | 0.1 | 0.4 | 10.4 |
| SEQ ID | 2.5 | μg/mL | 87.7 | 0.5 | 1.0 | 10.8 |
| NO. 2 | 5 | μg/mL | 81.3 | 0.4 | 2.8 | 15.5 |
| | 10 | μg/mL | 78.7 | 0.0 | 0.4 | 20.8 |
| | 20 | μg/mL | 71.5 | 0.0 | 0.4 | 28.1 |
| | 80 | μg/mL | 72.8 | 0.0 | 0.5 | 26.7 |
| Doxoru- | 10 | μM | 25.6 | 6.0 | 67.9 | 0.5 |
| bicin | 100 | μM | 1.3 | 1.9 | 96.6 | 0.1 |

TABLE 5
Fold increase in apoptosis in MDA-
MB-231 cells by Annexin-V staining

| | | | Fold increase (wrt control) | | |
|---|---|---|---|---|---|
| | | | LR | UR | UL |
| | | | Annex+ | Annex+ | Annex− |
| Sample (in MDA-MB-231) | | | 7-AAD− | 7-AAD+ | 7-AAD+ |
| SEQ ID | 2.5 | μg/mL | 15.8 | 22.0 | 4.6 |
| NO. 2 | 5 | μg/mL | 14.0 | 13.7 | 17.9 |
| | 10 | μg/mL | 11.6 | 15.8 | 29.4 |
| | 20 | μg/mL | 5.8 | 9.8 | 46.7 |
| | 80 | μg/mL | 10.6 | 12.8 | 37.1 |
| Doxorubicin | 10 | μM | 13.6 | 115.6 | — |
| | 100 | μM | — | 125.0 | — |

TABLE 6
Fold increase in apoptosis in PANC-1 cells by Annexin-V staining

| | | | Fold increase (wrt control) | | |
|---|---|---|---|---|---|
| | | | LR | UR | UL |
| | | | Annex+ | Annex+ | Annex− |
| Sample (in PANC-1) | | | 7-AAD− | 7-AAD+ | 7-AAD+ |
| SEQ ID | 2.5 | μg/mL | 5.0 | 2.5 | 1.0 |
| NO. 2 | 5 | μg/mL | 4.0 | 7.0 | 1.5 |
| | 10 | μg/mL | — | 1.0 | 2.0 |
| | 20 | μg/mL | — | 1.0 | 2.7 |
| | 80 | μg/mL | — | 1.3 | 2.6 |
| Doxorubicin | 10 | μM | 60.0 | 169.8 | — |
| | 100 | μM | — | 241.5 | — |

TABLE 7
Apoptotic effect of Lectin having the amino acid sequence
of SEQ ID NO. 2 in MDA-MB-231 cells by Cell cycle analysis

| | | | % Population of cells | | | |
|---|---|---|---|---|---|---|
| | | | Apoptotic | | | |
| Sample (in MDA-MB-231) | | | Sub(G0/G1) | G0/G1 | S | G2/M |
| Control | | | 2.46 | 64.40 | 8.18 | 16.79 |
| SEQ ID | 2.5 | μg/mL | 10.05 | 65.21 | 6.10 | 12.97 |
| NO. 2 | 5 | μg/mL | 5.75 | 61.99 | 8.15 | 15.40 |
| | 10 | μg/mL | 6.65 | 60.71 | 7.36 | 15.50 |
| | 20 | μg/mL | 15.43 | 55.66 | 6.55 | 13.35 |
| | 80 | μg/mL | 35.62 | 42.49 | 8.09 | 10.14 |
| Doxorubicin | 10 | μM | 11.09 | 40.26 | 22.70 | 18.52 |
| | 100 | μM | 92.02 | 5.64 | 1.17 | 0.83 |

TABLE 8
Apoptotic effect of Lectin having the amino acid sequence
of SEQ ID NO. 2 in PANC-1 cells by Cell cycle analysis

| | | | % Population of cells | | | |
|---|---|---|---|---|---|---|
| | | | Apoptotic Sub | | | |
| Sample (in PANC-1) | | | (G0/G1) | G0/G1 | S | G2/M |
| Control | | | 1.77 | 39.98 | 10.34 | 24.80 |
| SEQ ID NO. 2 | 2.5 | μg/mL | 1.60 | 38.89 | 9.89 | 24.83 |
| | 5 | μg/mL | 2.54 | 40.09 | 10.31 | 23.83 |
| | 10 | μg/mL | 4.23 | 44.43 | 10.15 | 23.92 |
| | 20 | μg/mL | 3.92 | 44.24 | 10.83 | 23.79 |
| | 80 | μg/mL | 6.21 | 39.63 | 9.58 | 27.41 |
| Doxorubicin | 10 | μM | 14.74 | 15.84 | 18.45 | 27.44 |
| | 100 | μM | 89.13 | 9.65 | 0.59 | 0.15 |

TABLE 9
Fold increase in apoptosis in MDA-
MB-231 cells by Cell cycle analysis

| | | | Fold Increase (wrt Control) |
|---|---|---|---|
| Sample (in MDA-MB-231) | | | Apoptotic Sub(G0/G1) |
| SEQ ID NO. 2 | 2.5 | μg/mL | 4.1 |
| | 5 | μg/mL | 2.3 |
| | 10 | μg/mL | 2.7 |
| | 20 | μg/mL | 6.3 |
| | 80 | μg/mL | 14.5 |
| Doxorubicin | 10 | μM | 5.0 |
| | 100 | μM | 41.5 |

TABLE 10
Fold increase in apoptosis in PANC-1 cells by Cell cycle analysis

| | | | Fold Increase (wrt Control) |
|---|---|---|---|
| Sample (in PANC-1) | | | Apoptotic Sub(G0/G1) |
| SEQ ID NO. 2 | 2.5 | μg/mL | — |
| | 5 | μg/mL | 1.4 |
| | 10 | μg/mL | 2.4 |
| | 20 | μg/mL | 2.2 |
| | 80 | μg/mL | 3.5 |
| Doxorubicin | 10 | μM | 7.9 |
| | 100 | μM | 47.9 |

Results for apoptotic effect of Recombinant lectin of SEQ ID NO 2 on MDA-MB-231 cell line demonstrated that it induced an increase in early, late apoptotic and necrotic cells. Additionally, enhancement of apoptotic (Sub G0/G1) cell population was also observed upon treatment of cells with SEQ ID NO. 2.

Results for Apoptotic effect of Recombinant Lectin having the amino acid sequence of SEQ ID NO. 2 in PANC-1 cell line induced an increase in late apoptotic and necrotic cells. Additionally, enhancement of apoptotic (SubG0/G1) cell population was also observed upon treatment of cells with SEQ ID NO. 2.

### Example 6: Anti-Angiogenic Effect of Lectin on Human Endothelial Cells

The cell line used for the study was EA.hy926 (Human endothelial cells) which was procured from National Centre for Cell Science, Pune (India). The cell line was maintained in DMEM+10% FBS (heat inactivated) at 37° C. with 5% CO2, and 95% humidity. The cells were counted using hemocytometer and plated in 96 well plates at the density of 5×10³ cells/well/180 μl of the growth medium. Following overnight incubation, cells were treated with recombinant lectin having amino acid sequence of SEQ ID NO.2 at concentrations ranging from about 2.5-100 μg/mL. Untreated cells with complete medium (10% FBS) served as complete medium control, cells with sera free medium served as SFM control and the cells treated with Paclitaxel served as positive control.

After 3 days of incubation, the effect of recombinant lectin having amino acid sequence of SEQ ID NO.2 on cell proliferation was determined by MTT assay. 20 μl of 5 mg/mL of MTT 3-(4,5-dimethythiazol-2-yl)-2,5-diphenyl tetrazolium bromide solution was added to all the wells followed by additional incubation for 3 h at 37° C. The supernatant was aspirated and 150 μl of DMSO was added to each well to dissolve formazan crystals. The absorbance of each well was then read at 540 nm using Synergy HT micro plate reader. The percentage cytotoxicity corresponding to each treatment was calculated.

### Cell Migration by Scratch Assay in Endothelial Cells

EA.hy296 cells were counted using hemocytometer and plated in 6-well plates at the density of 0.5×10⁶ cells/well. The cells were incubated overnight under growth conditions as described above so as to allow cell recovery and exponential growth. Following overnight incubation, a small linear scratch (representative wound) was created in the confluent monolayer (middle of the well) by gently scraping with sterile 200 μl micropipette tip. Photomicrographs of the scratch were taken at 0 h (Initial time point). Cells were rinsed with serum free DMEM and grouped for treatment in two different serum conditions:

### A: TI in Serum Free Medium (SFM)

Baseline control: Cells+DMEM
Positive/Validation control: Baseline control+Positive Control.
   Test: Baseline control+Test Item

### B: TI in DMEM+1% FBS

Baseline control: Cells+DMEM+1% FBS
Positive/Validation control: Baseline control+Positive Control.
Test: Baseline control+Test item

Photomicrographs of the scratch were taken at 24 h-72 h time points. The photomicrographs obtained in the above step were analyzed for quantitative assessment of area of wound closure using ImageJ tool software. Percentage migration with respect to untreated cells at different time points was calculated.

TABLE 11
Effect of SEQ ID NO: 2 on endothelial cell migration in SFM
Inhibition of migration with respect to untreated

| | | Treatment time (hours) | | |
|---|---|---|---|---|
| Sample | Concentration | 24 | 48 | 72 |
| Untreated | Untreated | 0 | 0 | 0 |
| Paclitaxel (nM) | 0.1 | 26.5 | 21.1 | 7.8 |
| | 1 | 28.5 | 36.8 | 32.7 |
| | 2.5 | 51.4 | 51.6 | 42.8 |
| | 5 | 43.7 | 45.2 | 47.3 |
| | 10 | 58.9 | 60.2 | 56.3 |
| SEQ ID No 2 | 20 | 74 | 81.1 | 82.4 |
| (μg/mL) | 40 | 80.5 | 82.3 | 82.2 |
| | 50 | 79.9 | 81 | 79.5 |
| | 60 | 82.0 | 77.3 | 76.0 |
| | 80 | 80.5 | 78.2 | 74.5 |
| | 100 | 55.7 | 73.2 | 71.5 |

TABLE 12
Effect of SEQ ID NO: 2 on endothelial cell migration in 1% FBS
Inhibition of migration with respect to untreated

Treatment time (hours)

| Sample | Concentration | 24 | 48 |
|---|---|---|---|
| Untreated | Untreated | 0 | 0 |
| Paclitaxel (nM) | 0.1 | −1.1 | −0.2 |
| | 1 | 7.8 | 2.1 |
| | 2.5 | 25.9 | 18.3 |
| | 5 | 25.0 | 10.6 |
| | 10 | 44.4 | 37.2 |
| SEQ ID NO: 2 | 20 | 0.8 | 5.4 |
| (μg/mL) | 40 | 28.5 | 35.3 |
| | 50 | 30.0 | 42.9 |
| | 60 | 50.1 | 63.5 |
| | 80 | 65.6 | 77.8 |
| | 100 | 73.8 | 73.1 |

### VEGF Secretion in Cancer Cells

VEGF secretion was studied in MDA-MB-231 (Human breast adenocarcinoma) and PANC-1 (Human pancreatic epitheloid carcinoma) which were procured from National Centre for Cell Science, Pune (India). The cell lines were maintained in DMEM+10% PBS (heat inactivated) at 37° C. with 5% CO2, and 95% humidity. Cells were counted and plated in 6-well plates for 24 h at a density of 0.5×10⁶ cells/well. The cells were incubated overnight under growth conditions as described above so as to allow cell recovery and exponential growth. Cells were treated with each test item at different concentrations for 24h in serum free media. Doxorubicin was used as positive control. Secreted levels of VEGF were determined in supernatants after 24 hours using Human VEGF ELISA kit (R&D systems) as per manufacturer's protocol. Change in VEGF levels corresponding to each treatment was calculated using the following formula:

% Change=[R−X)/R]*100

Where, X=absorbance of wells corresponding to treated cells
R=absorbance of untreated cells (cells maintained in growth medium)

### Results

The recombinant lectin having the amino acid sequence of SEQ ID NO.2 inhibited migration of endothelial cells (Table 13).

TABLE 13
Effect of Lectin having the amino acid sequence of SEQ ID
NO. 2 on proliferation of endothelial cells after 72 h

| | | | | % inhibition of |
|---|---|---|---|---|
| | | | | FBS stimulated |
| Sample | Concentration | Mean | SD | cell growth |
| DMEM alone | — | 0.66 | 0.00 | 58.89 |
| DMEM + 10% FBS | — | 1.60 | 0.02 | 0.00 |
| SEQ ID NO. 2 | 2.5 | 1.761 | 0.006 | −9.83 |
| (μg/mL) | 5 | 1.799 | 0.027 | −12.16 |
| | 10 | 1.870 | 0.012 | −16.59 |
| | 20 | 1.355 | 0.011 | 15.51 |
| | 40 | 1.135 | 0.012 | 29.22 |
| | 80 | 0.698 | 0.004 | 56.50 |
| | 100 | 0.665 | 0.008 | 58.53 |
| Paclitaxel | 1 | 0.93 | 0.01 | 42.03 |
| (nM) | 10 | 0.89 | 0.01 | 44.67 |
| | 100 | 0.57 | 0.01 | 64.46 |
| | 1000 | 0.45 | 0.00 | 71.65 |

The recombinant lectin having the amino acid sequence of SEQ ID NO.2 was observed to increase the level of VEGF in MDA-MB-231 cells at most concentrations tested across the range of 2.5-80 μg/mL, compared to the untreated control (Table 14). An increase in VEGF levels in the presence of the lectin was also observed for PANC-1 cells at all concentrations tested across the same concentration range (Table 15).

TABLE 14
Effect of Lectin having the amino acid sequence of
SEQ ID NO. 2 on VEGF release in MDA-MB-231 cells

| | | Average | VEGF | Percentage |
|---|---|---|---|---|
| | | absorbance | level | decrease in |
| Sample | Concentration | at 450 nM | (pg/mL) | VEGF levels |
| Untreated | | 0.796 | 463.3 | 0 |
| Doxorubicin | 10 | 0.176 | 49.9 | 89.2 |
| (μM) | 100 | 0.145 | 29.0 | 93.7 |
| SEQ ID NO. 2 | 2.5 | 0.970 | 579.3 | −25.0 |
| (μg/mL) | 5 | 0.763 | 441.0 | 4.8 |
| | 10 | 1.072 | 647.5 | −39.8 |
| | 20 | 1.552 | 967.3 | −108.8 |
| | 80 | 2.222 | 1413.9 | −205.2 |

TABLE 15
Effect of Lectin having the amino acid sequence
of SEQ ID NO. 2 on VEGF release in PANC-1 cells

| | | Average | VEGF | Percentage |
|---|---|---|---|---|
| | | absorbance | level | decrease in |
| Sample | Concentration | at 450 nM | (pg/mL) | VEGF levels |
| Untreated | | 1.633 | 1021.0 | 0 |
| Doxorubicin | 10 | 0.506 | 269.7 | 73.6 |
| (μM) | 100 | 0.483 | 254.6 | 75.1 |
| SEQ ID NO. 2 | 2.5 | 1.717 | 1077.5 | −5.5 |
| (μg/mL) | 5 | 1.821 | 1146.8 | −12.3 |
| | 10 | 2.085 | 1322.6 | −29.5 |
| | 20 | 2.202 | 1400.6 | −37.2 |
| | 80 | 2.006 | 1269.9 | −24.4 |

### Example 7: Evaluation of In-Vivo Anti-Angiogenesis Potential of Recombinant Lectin Having Amino Acid Sequence of SEQ NO. 2 Using Matrigel Plug Assay in c57bl/6 Mice

Healthy C57BL/6 mice were selected and grouped on the basis of body weight into five groups (G1-G4, n=7). Each mouse of group G2-G4 was subcutaneously injected at right flank region with 500 μl of matrigel containing 500 ng of FGF-2 (bFGF). Whereas mice of G1 group were subcutaneously injected at right flank region with 500 μl of matrigel only. In this assay, angiogenesis-inducing compounds such as bFGF was introduced into cold liquid Matrigel which, after subcutaneous injection, solidified and permits penetration by host cells and the formation of new blood vessels (neovascularization).

TABLE 16
Allocation of groups

| Group | Treatment | Dose, Route & Regimen | No. of Animals |
|---|---|---|---|
| G1 | Negative control | 10 mL/Kg, i.p., qdx15 | 7 |
| G2 | Positive control | 10 mg/Kg, i.p., qdx15 | 7 |
| G3 | Sunitinib | 55 mg/Kg, p.o., qdx15 | 7 |
| G4 | SEQ ID NO. 2 | 10 mg/Kg, i.p., qdx15 | 7 |

Required amount of test items were taken and added to an appropriate volume of Tris Buffered Saline (TBS) to achieve the required concentrations. The dose volume given to animal was 10 mL/Kg.

Daily cage-side observations were carried out to detect any clinical signs or mortality and recorded throughout the experiment period.

### Results:

### 1. Hemoglobin Content in the Matrigel Plug Section

Group G3 (Sunitinib, 55 mg/Kg) showed maximum reduction i.e. 59.2% in hemoglobin content in homogenate of Matrigel plug whereas Group G4 (recombinant lectin having amino acid sequence of SEQ ID No 2, 10 mg/Kg; qdx15) showed 23.6% reduction in hemoglobin content when compared to positive control (Table 17).

TABLE 17
Hemoglobin Content in the Matrigel Plug Section

| G1 | | G2 | | G3 | | G4 | |
|---|---|---|---|---|---|---|---|
| Negative control | | Positive control | | Sunitinib | | SEQ ID NO. 2 | |
| (10 mL/Kg, qdx15) | | (10 mL/Kg, qdx15) | | (55 mg/Kg, qdx15) | | (10 mg/Kg, qdx15) | |
| Mean | SEM | Mean | SEM | Mean | SEM | Mean | SEM |
| 5.67 | 1.15 | 53.76 | 11.74 | 21.92 | 3.11 | 41.10 | 7.00 |

### 2. Hematoxylin and Eosin (H&E) for Histological Observation

The severity of neovascularization drastically increased in the positive control group (G2) when compared to negative control group (G1) whereas neovascularization was marginally reduced in mice treated with recombinant lectin having amino acid sequence of SEQ ID NO. 2 at 10 mg/Kg, i.p., qdx15 (G4) (Table 18).

TABLE 18
Histological Observation

| Fibroblast | | | | | | | |
|---|---|---|---|---|---|---|---|
| Infiltration | | Neovascularization | | MN cell Infiltration | | Fat cell Infiltration | |
| Mean | SEM | Mean | SEM | Mean | SEM | Mean | SEM |

G1; Negative control, (10 mL/Kg, qdx15)

| | | | | | | | |
|---|---|---|---|---|---|---|---|
| 1.14 | 0.14 | 0.00 | 0.00 | 1.14 | 0.14 | 0.71 | 0.36 |

G2; Positive control, (10 mL/Kg, qdx15)

| | | | | | | | |
|---|---|---|---|---|---|---|---|
| 3.86 | 0.14 | 2.86 | 0.14 | 3.00 | 0.00 | 1.86 | 0.26 |

G3; Sunitinib, (55 mg/Kg, qdx15)

| | | | | | | | |
|---|---|---|---|---|---|---|---|
| 2.43 | 0.20 | 1.14 | 0.14 | 1.57 | 0.20 | 0.71 | 0.18 |

G4; recombinant lectin having amino acid
sequence of SEQ ID NO. 2, (10 mg/Kg, qdx15)

| | | | | | | | |
|---|---|---|---|---|---|---|---|
| 3.29 | 0.18 | 2.43 | 0.30 | 2.71 | 0.18 | 1.57 | 0.30 |

Hemoglobin content in Matrigel plug and neovascularization of Matrigel plug histology indicate that recombinant lectin having amino acid sequence of SEQ ID NO. 2 (10 mg/Kg; qdx15) showed anti-angiogenic activity when compared to positive control group using Matrigel plug assay in C57BL/6 mice.

### Example 8: Cytotoxicity, Apoptosis Studies on Brain Tumour Cytotoxicity

The in-vitro cytotoxic effects of recombinant lectin having amino acid sequence of SEQ ID NO. 2 was studied in a panel of Brain Tumor Cell lines consisting of Human Glioblastoma: LN-18, Human Glioblastoma: U251MG; Human Neuroblastoma: SH-SY-5Y; Human Meningioma: IOMM-Lee; Human Astrocytoma: U87MG; Rat C6 (Glioma) by MTT assay. The SEQ ID NO. 2 was provided as aqueous solution (12.17 mg/mL) and the stock solution of SEQ ID NO. 2 was diluted in serum free medium (SFM) at different concentrations corresponding to 10 fold high concentration. Cells were counted using hemocytometer and plated in 96-well plates and the cells were incubated overnight in 5% CO2 incubator at 37° C. After 24h of incubation, the cells were treated with different concentrations of SEQ ID NO. 2 ranging 2.5 μg/mL-100 μg/mL. Untreated cells were used as control. Cells treated with Everolimus and Doxorubicin were used as positive control. After 72 h of incubation, the effect of SEQ ID NO. 2 on cytotoxicity of cells was determined by MTT assay The plates were taken out and 20 μL of 5 mg/mL of MTT 3-(4, 5-dimethythiazol-2-yl)-2,5-diphenyl tetrazolium bromide solution was added to all the wells. Cells were incubated for 3 h at 37° C. The supernatant was aspirated and 150 μL of DMSO was added to each well to dissolve formazan crystals. The absorbance of each well was read at 540 nm using Synergy HT micro plate reader.

### Results

Percentage inhibition of cytotoxicity studies of SEQ ID NO. 2 against Human Glioblastoma: LN-18, Human Glioblastoma: U251MG; Human Neuroblastoma: SH-SY-5Y; Human Meningioma: IOMM-Lee; Human Astrocytoma: U87MG; Rat C6 (Glioma) has been tabulated in table 19.
SEQ ID NO. 2 demonstrated significant cytotoxicity in glioblastoma, meningioma and astrocytoma type of Brain Tumor
SEQ ID NO. 2 demonstrated good cytotoxicity in neuroblastoma type and glioma type cell type of Brain Tumor.
Cytotoxicity was observed as: LN18 (Glioblastoma)>U251MG (Glioblastoma)>IOMM-Lee (Meningioma)>U87MG (Astrocytoma)>C6 (Glioma)>SH-SY5Y (Neuroblastoma)
SEQ ID NO. 2 showed good selectivity in Glioblastoma type of Brain Tumor and moderate selectivity in Meningiomas.

TABLE 19
Percentage inhibition of cytotoxicity studies of SEQ ID
NO. 2 against Human Glioblastoma: LN-18, Human Glioblastoma:
U251MG; Human Neuroblastoma: SH-SY-5Y; Human Meningioma:
IOMM-Lee; Human Astrocytoma: U87MG; Rat C6 (Glioma)

| | % of cytotoxicity studies | | | | |
|---|---|---|---|---|---|
| | SEQ ID No 2 | Doxorubicin | Everolimus | | |
| | (2.5 μg/mL- | (0.1 Mm- | (0.05 μM- | IC50 Value | |
| Cell line | 100 μg/mL) | 100 μM) | 50 μM) | SEQ ID NO. 2 | |
| Glioblastoma | 39.6%-75.3% | 6.5%-47.7% | 3.5%-90.7% | 4.35 | μg/mL |
| (LN-18) | | | | | |
| Glioblastoma | 35.1%-74.1 | 11.2%-78.6% | 32.8% .71% | 11.05 | μg/mL |
| (U251MG) | | | | | |
| Meningioma | 15.4%-64.3% | 46.6%-54.5% | 44.7%-97.4% | 18.37 | μg/mL |
| (IOMM-Lee) | | | | | |
| Astrocytoma | 8.9%-51.8% | 46.6%-72.4% | 43.6%-88.0% | 98.88 | μg/mL |
| (U87MG) | | | | | |
| Glioma (C6) | 18.8-48.3% | 57.8%-86.3% | 50.3%-97.2% | >100 | μg/mL |
| Neuroblastoma | 10.3%-46.9% | 21.7%-61.7% | 29.7%-70.1% | >100 | μg/mL |
| (SH-SY5Y) | | | | | |

### Apoptosis

The evaluation of pro-apoptotic effect of SEQ ID NO. 2 in brain tumor cell lines U251MG & IOMM Lee was conducted. The cells were treated with SEQ ID NO. 2 at various concentrations around IC50 values. Resultant effect on apoptosis was determined via externalization of Phosphatidyl Serine (PS) on cell membrane by Annexin-V staining, mitochondrial membrane depolarization by JC-1 staining and cell cycle distribution analysis by Propidium Iodide (PI) staining. Increase in apoptotic markers in U251MG & IOMM-Lee cell lines reflected the pro-apoptotic potential of SEQ ID NO. 2 in brain tumor.
Externalization of PS on cell membrane by Annexin-V staining on U251MG cell line: SEQ ID NO. 2 (1 μg/mL-80 μg/mL) demonstrated an increase in Early and late apoptotic population by 3 fold-5 fold (p<0.001) and 2 fold-5 fold (p<0.05) as compared to Control (Untreated) respectively.
Externalization of PS on cell membrane by Annexin-V staining on IOMM-Lee cell line: SEQ ID NO. 2 (1 μg/mL-80 μg/mL) demonstrated an increase in early and late apoptotic population by 22 fold-28 fold (p<0.001) and 2 fold-5 fold as compared to Control respectively.
Mitochondrial membrane depolarization of U251MG cell line: SEQ ID NO. 2 (1 μg/mL-80 μg/mL) demonstrated an increase in mitochondrial membrane depolarization by 36.8%-60.9% (p<0.001) as compared to Control (Untreated).
Mitochondrial membrane depolarization of IOMM-Lee cell line: SEQ ID NO. 2 (1 μg/mL-80 μg/mL) demonstrated an increase in mitochondrial membrane depolarization by 16.1%-36.4% (p<0.001) as compared to Control (Untreated).
Increase in Sub(G0/G1) population in cell cycle analysis in U251MG cell line: SEQ ID NO. 2 (1 μg/mL-80 μg/mL) demonstrated an increase in apoptotic population; Sub(G0/G1) by 1.5 fold-4.3 fold (p<0.001) as compared to Control
Increase in Sub (G0/G1) population in cell cycle analysis in IOMM-Lee cell line: SEQ ID No 2 (1 μg/mL-500 μg/mL) demonstrated an increase in apoptotic population; Sub (G0/G1) by 1.1-35.3 fold (p<0.001) as compared to Control (Untreated).

### Example 9: Elucidation of Mechanism of Action of SEQ ID No 2 in Brain Tumor by Multiplex Analysis

Effect of SEQ ID No 2 on the expression of following 7 biomarkers were estimated in Human Meningioma cell line (IOMM-Lee) using multiplex analysis: TNF-alpha, VEGF, VEGFR2, HGF, HGFR/c-MET, PDGF-BB, and Contactin-1. Levels of another marker, Notch-1 were also estimated by ELISA in the same cell line. These 8 biomarkers play very crucial role in the pathogenesis and progression of Brain Tumor. Human meningioma (IOMM-Lee cells) were treated with SEQ ID No 2 at concentrations including IC50 for 48 h. Supernatants were collected and the levels of these 8 markers were investigated.

SEQ ID NO. 2 resulted in significant inhibition (p<0.01, p<0.001) of biomarkers (VEGF, VEGFR2, HGF, HGFR/c-MET, PDGF-BB, Notch-1) as compared to untreated control.

TNF-α and Contactin-1 levels were also inhibited by SEQ ID NO. 2.

TABLE 20
Percentage inhibition of SEQ ID NO. 2 on levels
of Brain Tumour biomarkers in IOMM-Lee

| | Seq. ID No 2 | | | | |
|---|---|---|---|---|---|
| | (1 μg/mL- | Doxorubicin | | Everolimus | |
| Bio Markers | 80 μg/mL) | 1 μM | 10 μM | 0.1 μM | 5 μM |
| TNF-α | 16.8%-42.3% | 67.8% | 42.3% | 42.3% | 100% |
| VEGF | 3.9%-49.3% | 53% | 93.4% | 45.8% | 53..3% |
| VEGFR2 | 17.8%-22.5% | 38.2% | 50.1% | 27.6% | 100% |
| HGF | 43.8%-93% | 45.3% | 84% | 59.2% | 80.8% |
| HGFR/c-MET | 5.5%-19.3% | 45.5% | 55.2% | 21.2% | 41% |
| PDGF-BB | 33.6%-100% | 12% | 100% | 1.5% | 67.9% |
| Contactin-1 | 0.3%-27.2% | 50% | 23.3% | 14.9% | 31.3% |
| Notch-1 | 81.2%-99.8% | 8% | 10.6% | 75.9% | — |

### Example 10: Cytotoxic Effect of SEQ ID NO. 2 in Cancer Cell Lines

### In Vitro Anti-Cancer Potential of Recombinant Lectin Having Amino Acid Sequence of SEQ ID 1 in Different Cell Lines by Calcein AM Assay

The purified recombinant lectin having amino acid sequence of SEQ ID NO. 2 was studied for its anti-cancer potential in different cell lines. The anti-cancer potential of recombinant lectin having amino acid sequence of SEQ ID 2 was evaluated in-vitro on 13 different cancer cell lines and 3 normal cells using Calcein AM assay.

All the cancer cell lines were procured from National Centre for Cell Science, Pune (India). The cell lines were maintained under conditions as described in Table 21 below. The cell lines were sub-cultured by trypsinization followed by splitting the cell suspension into fresh flasks and supplementing with fresh culture medium.

TABLE 21
Details of cell lines used in in vitro anti-cancer studies by Calcein AM assay.

| S No | Cell line/Cells Name | Growth medium |
|---|---|---|
| 1 | SW620 (Human Colorectal adenocarcinoma) | DMEM + 10% Heat Inactivated FBS |
| 2 | HT-29 (Human Colorectal adenocarcinoma) | DMEM + 10% Heat Inactivated FBS |
| 3 | PA-1 (Human Ovary carcinoma) | EMEM + 10% Heat Inactivated FBS |
| 4 | SKOV-3 (Human Ovary adenocarcinoma) | DMEM + 10% Heat Inactivated FBS |
| 5 | MCF-7 (Human breast adenocarcinoma) | DMEM + 10% Heat Inactivated FBS |
| 6 | A549 (Human lung carcinoma) | DMEM + 10% Heat Inactivated FBS |
| 7 | AGS (Human stomach adenocarcinoma) | EMEM + 10% Heat Inactivated FBS |
| 8 | ZR-75-1 (Human ductal carcinoma) | EMEM + 10% Heat Inactivated FBS |
| 9 | KB (Human papilloma carcinoma) | EMEM + 10% Heat Inactivated FBS |
| 10 | PC-3 (Human Prostate Cancer) | RPMI-1640 + 10% Heat Inactivated FBS |
| 11 | SiHa | EMEM + 10% Heat Inactivated FBS |
| | (Human cervix squamous cell carcinoma) | |
| 12 | THP-1 (Human acute monocytic leukemia) | RPMI-1640 + 0.05 mM beta- |
| | | mercaptoethanol + 10% Heat Inactivated |
| | | FBS |
| 13 | SK-Mel-28 (Human melanoma) | EMEM + 10% Heat Inactivated FBS |
| 14 | PBMCs (Peripheral blood mononuclear cells) | RPMI-1640 + 10% Heat Inactivated FBS |
| 15 | MCF-10A (Human mammary epithelial cells) | MEGM |
| 16 | Human Primary mammary epithelial (HMEC) | MEGM |
| | cells | |

All the cell lines were grown in specified media at 37° C. with 95% Humidity and % CO2. Doxorubicin was used as positive control. The stock solutions of recombinant lectin having amino acid sequence of SEQ ID 2 were prepared in dimethyl sulfoxide (DMSO) and used at final concentrations of 2.5, 5, 10, 20, 40 and 80 μg/mL. Similarly stock solution of Doxorubicin was prepared in DMSO and used at final concentration of 0.1, 1, 10 and 100 μM.

### Determination of Cytotoxic Activity

The cells were trypsinized, counted by Trypan blue method in Neuebauer's Chamber and plated in wells of flat bottom 96-well plate (dark walled plate, flat bottom) at the density corresponding to 10×10³ cells/well/180 μl medium.

Following overnight incubation, the cells were treated with test item (20 μL) at concentrations ranging from 2.5-80 μg/mL, so that the total volume in each well is 200 μL. The cells corresponding to positive control group were treated with Doxorubicin. The untreated cells served as negative control that does not receive any treatment.

The cells were incubated with the test items or positive control for a time period of 48h. Following incubation, the cell cytotoxicity was estimated using Calcein AM Cell Viability Assay Kit from R&D systems (Cat No. 4892-010-K).

Fluorescence was measured at 485 nm (excitation/528 run (emission) spectra using Synergy HT micro plate reader.

Percentage cytotoxicity wrt untreated cells was calculated using formula:

Cytotoxicity=[(RFUuntreated−RFUsample)/RFUuntreated]*100

RFU: relative fluorescence units

IC50 value was calculated using Graph-Pad Prism version 4.01 software.

The results demonstrated that recombinant lectin having amino acid sequence of SEQ ID NO. 2 showed cytotoxic effect on AGS (Human stomach adenocarcinoma), KB (Human cervical carcinoma), PA-1 (Human ovary carcinoma) and HT-29 (Human colorectal adenocarcinoma) cell lines with IC₅₀ values of 42.6, 18.4, 20.3, and 42.0 μg/mL respectively. Recombinant lectin having amino acid sequence of SEQ ID NO. 2 showed IC₅₀ value of >80 μg/mL for other cancer cells as that of normal cells. The results are summarized in table 22.

TABLE 22
Summary of in vitro anti-cancer potential of recombinant lectin having amino
acid sequence of SEQ ID 2 in different cell lines by Calcein AM assay

| | | Cytotoxicity at highest | | |
|---|---|---|---|---|
| | | concentration tested* | | IC₅₀ of |
| S. No. | Name of the cell line | SEQ 2 | Doxorubicin | SEQ ID2 |
| 1 | SW620 - (Human Colorectal adenocarcinoma) | 6.18 | 93.5 | >80 |
| 2 | HT-29 - (Human Colorectal | 59.8 | 75.0 | 42.0 |
| | adenocarcinoma) | | | |
| 3 | PA-1 - (Human Ovary carcinoma) | 72.0 | 82.3 | 20.3 |
| 4 | SKOV-3 - (Human Ovary adenocarcinoma) | −18.6 | 61.74 | >80 |
| 5 | MCF-7 - (Human breast adenocarcinoma) | −94.9 | 60.7 | >80 |
| 6 | A549 - (Human lung carcinoma) | 30.1 | 78.4 | >80 |
| 7 | AGS - (Human stomach adenocarcinoma) | 66.5 | 81.0 | 42.6 |
| 8 | ZR-75-1 - (Human ductal carcinoma) | −0.6 | 52.6 | >80 |
| 9 | KB - (Human cervical carcinoma) | 64.1 | 92.0 | 18.4 |
| 10 | PC-3 - (Human Prostate Cancer) | 35.0 | 75.3 | >80 |
| 11 | SiHa - (Human cervix squamous cell | 8.7 | 87.2 | >80 |
| | carcinoma) | | | |
| 12 | THP-1 - (Human acute monocytic leukemia) | 2.44 | 33.6 | >80 |
| 13 | SK-Mel-28 - (Human melanoma) | 18.2 | 65.9 | >80 |
| 14 | PBMCs - (Peripheral blood mononuclear cells) | 37.4 | 64.3 | >80 |
| 15 | MCF-10A - (Human mammary epithelial cells) | 43.5 | 93.8 | >80 |
| 16 | Human Primary mammary epithelial (HMEC) | 44.5 | 87.6 | >80 |
| | cells | | | |

*The concentration of recombinant lectin having amino acid sequence of SEQ ID 2 was 80 μg/mL and that of doxorubicin was 100 μM.

### In Vitro Anti-Cancer Potential of Recombinant Lectin Having Amino Acid Sequence of SEQ ID NO. 2 in Different Cell Lines by MTT Assay.

The purified recombinant lectin having amino acid sequence of SEQ ID NO. 2 was studied for its anti-cancer potential in different cell lines. The anti-cancer potential of recombinant lectin having amino acid sequence of SEQ ID 2 was evaluated in-vitro on 7 different cancer cell lines and one normal cell line using MTT assay.

All the cancer cell lines were procured from National Centre for Cell Science, Pune (India). Cell lines were maintained under conditions as mentioned in Table 23. The cell lines were sub-cultured by trypsinization followed by splitting the cell suspension into fresh flasks and supplementing with fresh culture medium.

TABLE 23
The details of cell lines used in in vitro
anti-cancer studies using Calcein AM assay.

| S No | Cell line/Cells Name | Growth medium |
|---|---|---|
| 1 | PANC-1 (Human pancreatic epitheloid carcinoma) | DMEM + 10% FBS |
| 2 | MIA PaCa-2 (Human pancreatic carcinoma) | DMEM + 10% FBS |
| 3 | MCF-7 (Human breast adenocarcinoma) | DMEM + 10% FBS |
| 4 | MDA-MB-231 (Human breast adenocarcinoma) | DMEM + 10% FBS |
| 5 | MDA-MB-453 (Human breast metastatic carcinoma) | DMEM + 10% FBS |
| 6 | T-47D (Human mammary gland ductal carcinoma) | DMEM + 10% FBS |
| 7 | T24 (Human Bladder Cancer) | McCoy's 5A + 10% FBS |
| 8 | PBMCs (Peripheral blood mononuclear cells) | RPMI-1640 + 10% FBS |

All the cell lines were grown in specified media at 37° C. with 95% Humidity and 5% CO2. Doxorubicin was used as positive control. The stock solutions of recombinant lectin having amino acid sequence of SEQ ID 2 were prepared in DMSO and used at final concentrations of 2.5 μg/mL, 5 μg/mL, 10 μg/mL, 20 μg/mL, 40 μg/mL and 80 μg/mL. Similarly stock solution of Doxorubicin was prepared in DMSO and used at final concentration of 0.1 μM, 1 μM, 10 μM, 25 μM, 50 μM and 75 μM.

### Determination of Cytotoxic Activity

The cells were trypsinized, counted by Trypan blue method in Neuebauer's Chamber and plated in wells of flat bottom 96-well plate at the density corresponding to 10×10³ cells/well/200 μL medium.

Following overnight incubation, the media in the plate was replenished with 180 μL/well and then the cells were treated with 20 μL of each test item at concentrations ranging from 2.5 μg/mL-80 μg/mL in triplicates, so that the total volume in each well is 200 μL.

The cells corresponding to positive control group were treated with Doxorubicin. The untreated cells served as negative control. The cells were incubated with the test items or positive control for a time period of 48h. Following incubation, the cell cytotoxicity was estimated using MTT assay. Absorbance was measured at 540 nm. The percentage cytotoxicity corresponding to each treatment was calculated using the formula:

% Cytotoxicity=(1−X/R)*100

Where X=absorbance of wells corresponding to treated cells
R=absorbance of untreated cells (cells maintained in growth medium only)

IC50 value was calculated using Graph-Pad Prism version 4.01 software.

The results demonstrated that recombinant lectin having amino acid sequence of

SEQ ID NO. 2 showed cytotoxic effect on PANC-1 (Human pancreatic epitheloid carcinoma), MDA-MB-231 (Human breast adenocarcinoma) and T24 (Human Bladder Cancer) cell lines with IC₅₀ values of 24.3 μg/mL, 9.7 μg/mL, and 10.4 μg/mL respectively. Recombinant lectin of SEQ ID NO. 2 showed IC50 value of >80 μg/ml for all other cell lines. The results are summarized in Table 24.

TABLE 24
Summary of cell based in vitro cytotoxicity/anti-proliferation assay

| | | Cytotoxicity at highest | | |
|---|---|---|---|---|
| | | concentration tested* | | IC₅₀ of |
| S No | Name of the cell line | SEQ 2 | Doxorubicin | SEQ ID2 |
| 1 | PANC-1 - (Human pancreatic epitheloid | 60.8 | 65.1 | 24.3 |
| | carcinoma) | | | |
| 2 | MIA PaCa-2 - (Human pancreatic carcinoma) | 47.5 | 63.9 | >80 |
| 3 | MCF-7 - (Human breast adenocarcinoma) | 28 | 64.9 | >80 |
| 4 | MDA-MB-231 - (Human breast | 60.9 | 50.2 | 9.7 |
| | adenocarcinoma) | | | |
| 5 | MDA-MB-453 - (Human breast metastatic | 46.6 | 71.4 | >80 |
| | carcinoma) | | | |
| 6 | T-47D - (Human mammary gland ductal | 28.1 | 51.1 | >80 |
| | carcinoma) | | | |
| 7 | T24 - (Human Bladder Cancer) | 93.1 | 54.4 | 10.4 |
| 8 | PBMCs - (Peripheral blood mononuclear cells) | 2.1 | 32.0 | >80 |

*The concentration of recombinant lectin having amino acid sequence of SEQ ID NO. 2 was 80 μg/ml and that of doxorubicin was 75 μM.

1. A recombinant lectin for use in a method of treatment of cancer by inhibiting angiogenesis or by inducing apoptosis in a subject, the method comprising administration of a therapeutically effective amount of the recombinant lectin.

2. (canceled)

3. The recombinant lectin of claim 1, wherein the cancer is carcinoma.

4. The recombinant lectin of claim 3, wherein the cancer is adenocarcinoma or squamous cell carcinoma.

5. The recombinant lectin of claim 4, wherein adenocarcinoma is selected from oesophageal, pancreatic, prostate, cervical, breast, colon or colorectal, lung, bile duct, vaginal, urachus, or stomach adenocarcinoma.

6. The recombinant lectin of claim 4, wherein squamous cell carcinoma is selected from skin, lung, oral, thyroid, oesophagus, vaginal, cervical, ovarian, head and/or neck, prostate, or bladder squamous cell carcinoma

7. The recombinant lectin of claim 3, wherein the cancer is brain cancer.

8. The recombinant lectin of claim 1, wherein the recombinant lectin is represented by an amino acid sequence having at least 60% identity to SEQ ID NO. 1.

9. The recombinant lectin of claim 8, wherein the amino acid sequence has at least 70%, 80%, 90%, 95%, 96%, 97%, 98%, or 99% homology to SEQ ID NO. 1.

10. The recombinant lectin of claim 8, wherein the amino acid sequence is selected from SEQ ID NO. 2, SEQ ID NO. 3, or SEQ ID NO. 4.

11. The recombinant lectin of claim 1, wherein the effective concentration of the recombinant lectin is from 0.1 μg/mL to about 200 μg/mL.

12. The recombinant lectin of claim 1, wherein the therapeutically effective dose of the recombinant lectin is from 0.1 mg/Kg to 100 mg/Kg of a body weight of a subject.

13. The recombinant lectin of claim 1, wherein the recombinant lectin inhibits migration and/or proliferation of endothelial cells.

14. The recombinant lectin of claim 1, wherein the recombinant lectin modulates VEGF secretion.

15. The recombinant lectin of claim 1, wherein the recombinant lectin reduces hemoglobin content and neovascularization in cancer cells.

16. The recombinant lectin of claim 1, wherein the recombinant lectin modulates one or more signaling pathways selected from: ATF-2, ERK1/2; JNK; MEK-1; P90RSK; STAT-3; p53; MMPs; HGF; C-kit; Her-2; GMSCF; IL-6; IL-8; p38/MAPK; PDGF; TNFR; MPO; Galectin-3; Fol-1; CD40L; Angiopoietin-2; Kalikrein-5; Osteopontin; TNF-α; Endoglin; CCR5; TRAIL via FADD and caspase-3, Leptin; MAPK/EGFR/Ras/Raf; ADBR1; IL-4/STATE; NF-KB; TNF-α/JNK, PKC/CA2+; and PI3K/AKT/FOXO3.

17. The recombinant lectin of claim 1, wherein the recombinant lectin induces early and late stage of apoptosis in the cancer cells.

18. A method of preventing angiogenesis in cancer cells or inducing apoptosis of tumor cells using a recombinant lectin protein.

19. (canceled)

20. (canceled)

21. (canceled)

22. (canceled)

23. (canceled)

24. (canceled)

25. (canceled)

26. (canceled)

27. The method of claim 18, wherein the cancer cells comprise carcinoma cancer cells.

28. The method of claim 18, wherein the cancer cells comprise adenocarcinoma or squamous cell carcinoma cancer cells or brain cancer cells.

29. A pharmaceutical composition for use in a method of treatment of cancer comprising a recombinant lectin protein and a pharmaceutically acceptable excipient, wherein:
the recombinant lectin protein inhibits angiogenesis or induces apoptosis in a subject with administration of a therapeutically effective amount of the recombinant lectin protein; and
the pharmaceutical composition inhibits angiogenesis in the cancer cells or induces apoptosis in cancer cells.
